(19) United States
(12) Patent Application Publication
(10) Pub. No.: US 2025/0076337 A1
(43) Pub. Date: Mar. 6, 2025

(54) METHOD AND ELECTRONIC DEVICE FOR DETERMINING USER ACTIVITY STATE ON BASIS OF PLURALITY OF ELECTRONIC DEVICES

(71) Applicant: Samsung Electronics Co., Ltd., Suwon-si (KR)

(72) Inventors: Hyeonseong KIM, Suwon-si (KR); Jeongmin Park, Suwon-si (KR)

(73) Assignee: Samsung Electronics Co., Ltd., Suwon-si (KR)

(21) Appl. No.: 18/952,444

(22) Filed: Nov. 19, 2024

Related U.S. Application Data
(63) Continuation of application No. PCT/KR2023/002262, filed on Feb. 16, 2023.

(30) Foreign Application Priority Data
May 20, 2022 (KR) 10-2022-0061852
Jun. 30, 2022 (KR) 10-2022-0080249

(51) Int. Cl. G01P 13/00

(52) U.S. Cl. CPC G01P 13/00

(57) ABSTRACT

A system for determining an activity state of a user includes: a first electronic device; and a second electronic device, where the first electronic device is configured to: request the second electronic device to identify a first activity state detected as an activity state of a user in first electronic device, receive a second activity state corresponding to an identification result of identifying the first activity state in the second electronic device received from the second electronic device in response to the request and update the activity state of the user based on the second activity state, and where the second electronic device is configured to: based on receiving the request for identifying the first activity state from the first electronic device, identify whether the activity state of the user is the first activity state and transmit the second activity state corresponding to the identification result to the first electronic device.

### CROSS-REFERENCE TO RELATED APPLICATION(S)

This application is a continuation of International Application No. PCT/KR2023/002262, filed on Feb. 16, 2023, in the Korean Intellectual Property Receiving Office, which is based on and claims priority to Korean Patent Applications No. 10-2022-0061852, filed on May 20, 2022 and No. 10-2022-0080249, filed on Jun. 30, 2022, in the Korean Intellectual Property Office, the disclosures of which are incorporated by reference herein in their entireties.

### BACKGROUND

### 1. Field

The disclosure relates to a method and electronic device for determining the activity state of a user based on a plurality of electronic devices.

### 2. Description of Related Art

Recent electronic devices come in various form factors for user convenience purposes and in reduced size for easy carrying.

As a function capable of detecting the user's risk state is included in execution of various services or functions of an electronic device, a contact function of contacting a designated contact point when the user's risk state is detected may be automatically performed.

For example, when the user's fall is detected using a fall detection algorithm included in the electronic device, an emergency or medical alert message may be transmitted to a designated emergency contact.

In an electronic device, a fall detection algorithm for detecting the user's fall is separately operated in the user's activity state, e.g., a walking state and a biking state. Accordingly, when the electronic device misrecognizes the user's activity state, a wrong fall algorithm operates, causing false or non-recognition of the fall.

For example, when the user is pulling a cart or a stroller while putting on a wearable electronic device, the wearable electronic device may mis-recognize it as the biking situation by an acceleration sensor for detecting the vibration pattern of the road surface while biking although the user is walking. Further, when the user drives a car, the wearable electronic device may mis-recognize it as the biking situation by a gyro sensor for recognizing the bicycle handling motion although the user is driving a car.

### SUMMARY

Provided are an electronic device and an operation method thereof, which may determine the activity state of a user with higher accuracy using a plurality of electronic devices.

However, aspects of the disclosure are not limited to the foregoing aspects but rather may be expanded in various manners without departing from the spirit and scope of the disclosure. Additional aspects will be set forth in part in the description which follows and, in part, will be apparent from the description, or may be learned by practice of the presented embodiments.

According to an aspect of the disclosure, a system for determining an activity state of a user may include: a first electronic device; and a second electronic device, where the first electronic device is configured to: request the second electronic device to identify a first activity state detected as an activity state of a user in first electronic device, receive a second activity state corresponding to an identification result of identifying the first activity state in the second electronic device received from the second electronic device in response to the request, and update the activity state of the user based on the second activity state, and where the second electronic device is configured to: based on receiving the request for identifying the first activity state from the first electronic device, identify whether the activity state of the user is the first activity state and transmit the second activity state corresponding to the identification result to the first electronic device.

The first electronic device may include a wearable electronic device.

The first electronic device may be further configured to detect the first activity state based on at least one of a sensor module or a communication module included in the first electronic device.

The first electronic device may be further configured to update the activity state of the user to the first activity state based on the second activity state received from the second electronic device corresponding to the first activity state.

The first electronic device may be further configured to update the activity state of the user to the second activity state based on the second activity state received from the second electronic device being different from the first activity state.

The second electronic device may be further configured to: based on identifying that a walking step occurs and the second electronic device is carried by the user using a sensor module of the second electronic device, detect a walking state of the user as the second activity state; based on identifying that the second electronic device is located indoor and a moving speed of the second electronic device meets a threshold speed range using at least one of the sensor module or communication module of the second electronic device, detect a biking state as the second activity state; and detect at least one of vibration pattern information about a vehicle, vehicle engine output sound information, or magnetic pattern information corresponding to a traveling state of the vehicle using at least one of the sensor module or the communication module of the second electronic device and, based on identifying that the moving speed of the second electronic device exceeds a second threshold speed, detect a vehicle boarding state as the second activity state.

According to an aspect of the disclosure, a first electronic device may include: a sensor module; a communication module; at least one processor; and memory storing instructions that, when executed by the at least one processor individually or collectively, cause the electronic device to: detect a first activity state indicating an activity state of a user based on at least one of the sensor module or the communication module; receive a second activity state corresponding to a result of identifying the first activity state in a first electronic device from the first electronic device through the communication module in response to a request for identifying the first activity state; and update the activity state of the user based on the second activity state received from the first electronic device.

wherein the instructions, when executed by the at least one processor individually or collectively, cause the wearable electronic device to: update the activity state of the user to the first activity state based on the second activity state received from the first electronic device corresponding to the first activity state; and update the activity state of the user to the second activity state based on the second activity state received from the first electronic device being different from the first activity state.

According to an aspect of the disclosure, a second electronic device may include: a communication module; sensor module; at least one processor; and memory storing instructions that, when executed by the at least one processor individually or collectively, cause the electronic device to: based on receiving a request for identifying a first activity state from a first electronic device, detect a second activity state corresponding to a result of identifying whether an activity state of a user is the first activity state based on at least one of the sensor module or the communication module; and transmit the second activity state to the first electronic device through the communication module.

wherein the instructions, when executed by the at least one processor individually or collectively, cause the wearable electronic device to: based on identifying that a walking step occurs and the electronic device is carried by the user using the sensor module, detect a walking state of the user as the second activity state; based on identifying that the electronic device is located indoor and a moving speed of the second electronic device meets a threshold speed range using at least one of the sensor module or the communication module, detect a biking state as the second activity state; detect at least one of vibration pattern information about a vehicle, magnetic pattern information corresponding to a traveling state of the vehicle, or vehicle engine output sound information using at least one of the sensor module or the communication module; and based on identifying that the moving speed of the electronic device exceeds a second threshold speed, detect a vehicle boarding state as the second activity state.

According to an aspect of the disclosure, a method for operating an electronic device may include: requesting, by a first electronic device, a second electronic device to identify a first activity state detected as an activity state of a user; identifying, by the second electronic device, whether the activity state of the user is the first activity state based on receiving the request for identifying the first activity state from the first electronic device; transmitting, by the second electronic device, the second activity state corresponding to the identification result to the first electronic device; and updating, by the first electronic device, the activity state of the user based on a second activity state corresponding to an identification result, a result of identifying the first activity state in the second electronic device received from the second electronic device in response to the request.

The method may further include detecting, by the first electronic device, the first activity state based on at least one of a sensor module or a communication module included in the first electronic device.

The method may further include updating, by the first electronic device, the activity state of the user to the first activity state based on the second activity state received from the first electronic device corresponding to the first activity state.

The method may further include: updating, by the first electronic device, the activity state of the user to the second activity state based on the second activity state received from the first electronic device being different from the first activity state.

The method may further include: based on identifying that a walking step occurs and the second electronic device is carried by the user using a sensor module of the second electronic device, detecting, by the second electronic device, the a walking state of the user as the second activity state; based on identifying that the second electronic device is located indoor and a moving speed of the second electronic device meets a threshold speed range using at least one of the sensor module or communication module of the second electronic device, detecting, by the second electronic device, a biking state as the second activity state; and detecting, by the second electronic device, at least one of vibration pattern information about a vehicle, magnetic pattern information corresponding to a traveling state of the vehicle, or vehicle engine output sound information using at least one of the sensor module or the communication module of the second electronic device and, based on identifying that the moving speed of the second electronic device exceeds a second threshold speed, detect a vehicle boarding state as the second activity state.

### BRIEF DESCRIPTION OF THE DRAWINGS

The above and other aspects, features, and advantages of certain embodiments of the present disclosure will be more apparent from the following description taken in conjunction with the accompanying drawings, in which:

FIG. 1 is a view illustrating an electronic device in a network environment according to an embodiment of the disclosure;

FIG. 2 is a view illustrating a system capable of determining a user's activity state using a plurality of electronic devices according to an embodiment of the disclosure;

FIG. 3 is a block diagram illustrating a first electronic device according to an embodiment of the disclosure;

FIG. 4 is a block diagram illustrating a second electronic device according to an embodiment of the disclosure;

FIG. 5 is a flowchart illustrating an operation of determining a user's activity state using a plurality of electronic devices according to an embodiment of the disclosure;

FIG. 6 is a flowchart illustrating an operation of determining a user's activity state using a plurality of electronic devices according to an embodiment of the disclosure;

FIG. 7 is a flowchart illustrating an operation of determining a user's activity state in a second electronic device according to an embodiment of the disclosure;

FIG. 8A and FIG. 8B are flowcharts illustrating an operation of determining a user's activity state using a plurality of electronic devices according to an embodiment of the disclosure; and

FIG. 9 is a flowchart illustrating an operation of determining a user's activity state in a first electronic device according to an embodiment of the disclosure.

### DETAILED DESCRIPTION

Hereinafter, example embodiments of the disclosure will be described in detail with reference to the accompanying drawings. The same reference numerals are used for the same components in the drawings, and redundant descriptions thereof will be omitted. The embodiments described herein are example embodiments, and thus, the disclosure is not limited thereto and may be realized in various other forms. It is to be understood that singular forms include plural referents unless the context clearly dictates otherwise. The terms including technical or scientific terms used in the disclosure may have the same meanings as generally understood by those skilled in the art.

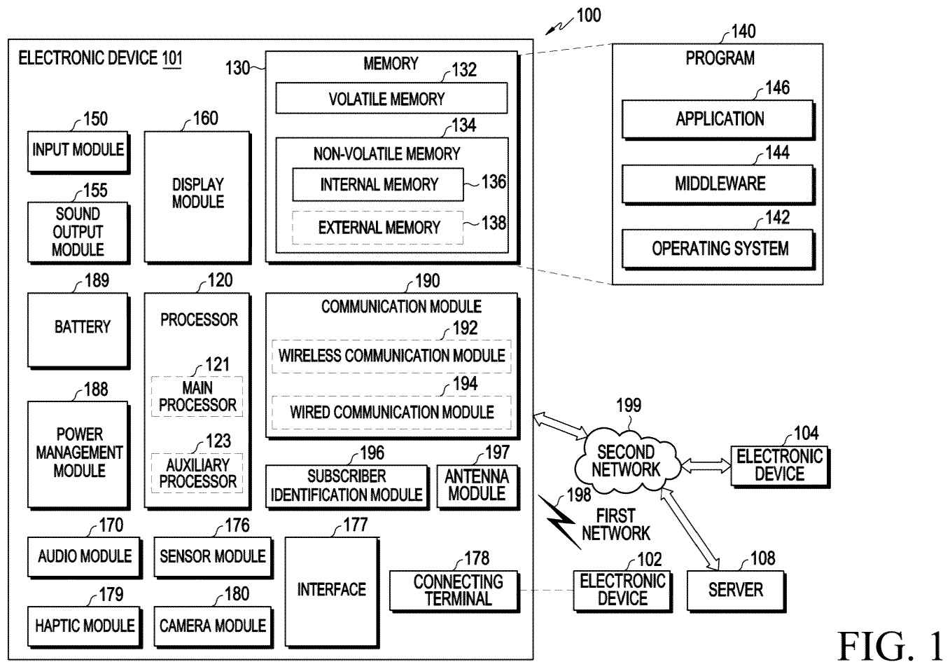

FIG. 1 is a block diagram illustrating an electronic device 101 in a network environment 100 according to an embodiment of the disclosure. Referring to FIG. 1, the electronic device 101 in the network environment 100 may communicate with at least one of an electronic device 102 via a first network 198 (e.g., a short-range wireless communication network), or an electronic device 104 or a server 108 via a second network 199 (e.g., a long-range wireless communication network). According to an embodiment, the electronic device 101 may communicate with the electronic device 104 via the server 108. According to an embodiment, the electronic device 101 may include a processor 120, memory 130, an input module 150, a sound output module 155, a display module 160, an audio module 170, a sensor module 176, an interface 177, a connecting terminal 178, a haptic module 179, a camera module 180, a power management module 188, a battery 189, a communication module 190, a subscriber identification module (SIM) 196, or an antenna module 197. In an embodiment, at least one (e.g., the connecting terminal 178) of the components may be omitted from the electronic device 101, or one or more other components may be added in the electronic device 101. According to an embodiment, some (e.g., the sensor module 176, the camera module 180, or the antenna module 197) of the components may be integrated into a single component (e.g., the display module 160).

The processor 120 may execute, for example, software (e.g., a program 140) to control at least one other component (e.g., a hardware or software component) of the electronic device 101 coupled with the processor 120, and may perform various data processing or computation. According to an embodiment, as at least part of the data processing or computation, the processor 120 may store a command or data received from another component (e.g., the sensor module 176 or the communication module 190) in volatile memory 132, process the command or the data stored in the volatile memory 132, and store resulting data in non-volatile memory 134. According to an embodiment, the processor 120 may include a main processor 121 (e.g., a central processing unit (CPU) or an application processor (AP)), or an auxiliary processor 123 (e.g., a graphics processing unit (GPU), a neural processing unit (NPU), an image signal processor (ISP), a sensor hub processor, or a communication processor (CP)) that is operable independently from, or in conjunction with, the main processor 121. For example, when the electronic device 101 includes the main processor 121 and the auxiliary processor 123, the auxiliary processor 123 may be configured to use lower power than the main processor 121 or to be specified for a designated function. The auxiliary processor 123 may be implemented as separate from, or as part of the main processor 121.

The auxiliary processor 123 may control at least some of functions or states related to at least one component (e.g., the display module 160, the sensor module 176, or the communication module 190) among the components of the electronic device 101, instead of the main processor 121 while the main processor 121 is in an inactive (e.g., sleep) state, or together with the main processor 121 while the main processor 121 is in an active state (e.g., executing an application). According to an embodiment, the auxiliary processor 123 (e.g., an image signal processor or a communication processor) may be implemented as part of another component (e.g., the camera module 180 or the communication module 190) functionally related to the auxiliary processor 123. According to an embodiment, the auxiliary processor 123 (e.g., the neural processing unit) may include a hardware structure specified for artificial intelligence model processing. The artificial intelligence model may be generated via machine learning. Such learning may be performed, e.g., by the electronic device 101 where the artificial intelligence is performed or via a separate server (e.g., the server 108). Learning algorithms may include, but are not limited to, e.g., supervised learning, unsupervised learning, semi-supervised learning, or reinforcement learning. The artificial intelligence model may include a plurality of artificial neural network layers. The artificial neural network may be a deep neural network (DNN), a convolutional neural network (CNN), a recurrent neural network (RNN), a restricted Boltzmann machine (RBM), a deep belief network (DBN), a bidirectional recurrent deep neural network (BRDNN), deep Q-network or a combination of two or more thereof but is not limited thereto. The artificial intelligence model may, additionally or alternatively, include a software structure other than the hardware structure.

The memory 130 may store various data used by at least one component (e.g., the processor 120 or the sensor module 176) of the electronic device 101. The various data may include, for example, software (e.g., the program 140) and input data or output data for a command related thereto. The memory 130 may include the volatile memory 132 or the non-volatile memory 134.

The program 140 may be stored in the memory 130 as software, and may include, for example, an operating system (OS) 142, middleware 144, or an application 146.

The input module 150 may receive a command or data to be used by other component (e.g., the processor 120) of the electronic device 101, from the outside (e.g., a user) of the electronic device 101. The input module 150 may include, for example, a microphone, a mouse, a keyboard, keys (e.g., buttons), or a digital pen (e.g., a stylus pen).

The sound output module 155 may output sound signals to the outside of the electronic device 101. The sound output module 155 may include, for example, a speaker or a receiver. The speaker may be used for general purposes, such as playing multimedia or playing record. The receiver may be used for receiving incoming calls. According to an embodiment, the receiver may be implemented as separate from, or as part of the speaker.

The display module 160 may visually provide information to the outside (e.g., a user) of the electronic device 101. The display 160 may include, for example, a display, a hologram device, or a projector and control circuitry to control a corresponding one of the display, hologram device, and projector. According to an embodiment, the display 160 may include a touch sensor configured to detect a touch, or a pressure sensor configured to measure the intensity of a force generated by the touch.

The audio module 170 may convert a sound into an electrical signal and vice versa. According to an embodiment, the audio module 170 may obtain the sound via the input module 150, or output the sound via the sound output module 155 or a headphone of an external electronic device (e.g., an electronic device 102) directly (e.g., wiredly) or wirelessly coupled with the electronic device 101.

The sensor module 176 may detect an operational state (e.g., power or temperature) of the electronic device 101 or an environmental state (e.g., a state of a user) external to the electronic device 101, and then generate an electrical signal or data value corresponding to the detected state. According to an embodiment, the sensor module 176 may include, for example, a gesture sensor, a gyro sensor, an atmospheric pressure sensor, a magnetic sensor, an accelerometer, a grip sensor, a proximity sensor, a color sensor, an infrared (IR) sensor, a biometric sensor, a temperature sensor, a humidity sensor, or an illuminance sensor.

The interface 177 may support one or more specified protocols to be used for the electronic device 101 to be coupled with the external electronic device (e.g., the electronic device 102) directly (e.g., wiredly) or wirelessly. According to an embodiment, the interface 177 may include, for example, a high definition multimedia interface (HDMI), a universal serial bus (USB) interface, a secure digital (SD) card interface, or an audio interface.

A connecting terminal 178 may include a connector via which the electronic device 101 may be physically connected with the external electronic device (e.g., the electronic device 102). According to an embodiment, the connecting terminal 178 may include, for example, an HDMI connector, a USB connector, an SD card connector, or an audio connector (e.g., a headphone connector).

The haptic module 179 may convert an electrical signal into a mechanical stimulus (e.g., a vibration or motion) or electrical stimulus which may be recognized by a user via his tactile sensation or kinesthetic sensation. According to an embodiment, the haptic module 179 may include, for example, a motor, a piezoelectric element, or an electric stimulator.

The camera module 180 may capture a still image or moving images. According to an embodiment, the camera module 180 may include one or more lenses, image sensors, image signal processors, or flashes.

The power management module 188 may manage power supplied to the electronic device 101. According to an embodiment, the power management module 188 may be implemented as at least part of, for example, a power management integrated circuit (PMIC).

The battery 189 may supply power to at least one component of the electronic device 101. According to an embodiment, the battery 189 may include, for example, a primary cell which is not rechargeable, a secondary cell which is rechargeable, or a fuel cell.

The communication module 190 may support establishing a direct (e.g., wired) communication channel or a wireless communication channel between the electronic device 101 and the external electronic device (e.g., the electronic device 102, the electronic device 104, or the server 108) and performing communication via the established communication channel. The communication module 190 may include one or more communication processors that are operable independently from the processor 120 (e.g., the application processor (AP)) and supports a direct (e.g., wired) communication or a wireless communication. According to an embodiment, the communication module 190 may include a wireless communication module 192 (e.g., a cellular communication module, a short-range wireless communication module, or a global navigation satellite system (GNSS) communication module) or a wired communication module 194 (e.g., a local area network (LAN) communication module or a power line communication (PLC) module). A corresponding one of these communication modules may communicate with the external electronic device 104 via a first network 198 (e.g., a short-range communication network, such as Bluetooth™, wireless-fidelity (Wi-Fi) direct, or infrared data association (IrDA)) or a second network 199 (e.g., a long-range communication network, such as a legacy cellular network, a 5G network, a next-generation communication network, the Internet, or a computer network (e.g., local area network (LAN) or wide area network (WAN)). These various types of communication modules may be implemented as a single component (e.g., a single chip), or may be implemented as multi components (e.g., multi chips) separate from each other. The wireless communication module 192 may identify or authenticate the electronic device 101 in a communication network, such as the first network 198 or the second network 199, using subscriber information (e.g., international mobile subscriber identity (IMSI)) stored in the subscriber identification module 196.

The wireless communication module 192 may support a 5G network, after a 4G network, and next-generation communication technology, e.g., new radio (NR) access technology. The NR access technology may support enhanced mobile broadband (eMBB), massive machine type communications (mMTC), or ultra-reliable and low-latency communications (URLLC). The wireless communication module 192 may support a high-frequency band (e.g., the mmWave band) to achieve, e.g., a high data transmission rate. The wireless communication module 192 may support various technologies for securing performance on a high-frequency band, such as, e.g., beamforming, massive multiple-input and multiple-output (massive MIMO), full dimensional MIMO (FD-MIMO), array antenna, analog beam-forming, or large scale antenna. The wireless communication module 192 may support various requirements specified in the electronic device 101, an external electronic device (e.g., the electronic device 104), or a network system (e.g., the second network 199). According to an embodiment, the wireless communication module 192 may support a peak data rate (e.g., 20 Gbps or more) for implementing eMBB, loss coverage (e.g., 164 dB or less) for implementing mMTC, or U-plane latency (e.g., 0.5 ms or less for each of downlink (DL) and uplink (UL), or a round trip of 1 ms or less) for implementing URLLC.

The antenna module 197 may transmit or receive a signal or power to or from the outside (e.g., the external electronic device). According to an embodiment, the antenna module 197 may include one antenna including a radiator formed of a conductor or conductive pattern formed on a substrate (e.g., a printed circuit board (PCB)). According to an embodiment, the antenna module 197 may include a plurality of antennas (e.g., an antenna array). In this case, at least one antenna appropriate for a communication scheme used in a communication network, such as the first network 198 or the second network 199, may be selected from the plurality of antennas by, e.g., the communication module 190. The signal or the power may then be transmitted or received between the communication module 190 and the external electronic device via the selected at least one antenna. According to an embodiment, other parts (e.g., radio frequency integrated circuit (RFIC)) than the radiator may be further formed as part of the antenna module 197.

According to an embodiment, the antenna module 197 may form a mm Wave antenna module. According to an embodiment, the mmWave antenna module may include a printed circuit board, a RFIC disposed on a first surface (e.g., the bottom surface) of the printed circuit board, or adjacent to the first surface and capable of supporting a designated high-frequency band (e.g., the mmWave band), and a plurality of antennas (e.g., array antennas) disposed on a second surface (e.g., the top or a side surface) of the printed circuit board, or adjacent to the second surface and capable of transmitting or receiving signals of the designated high-frequency band.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

According to an embodiment, commands or data may be transmitted or received between the electronic device 101 and the external electronic device 104 via the server 108 coupled with the second network 199. The external electronic devices 102 or 104 each may be a device of the same or a different type from the electronic device 101. According to an embodiment, all or some of operations to be executed at the electronic device 101 may be executed at one or more of the external electronic devices 102, 104, or 108. For example, if the electronic device 101 should perform a function or a service automatically, or in response to a request from a user or another device, the electronic device 101, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and transfer an outcome of the performing to the electronic device 101. The electronic device 101 may provide the outcome, with or without further processing of the outcome, as at least part of a reply to the request. To that end, a cloud computing, distributed computing, mobile edge computing (MEC), or client-server computing technology may be used, for example. The electronic device 101 may provide ultra low-latency services using, e.g., distributed computing or mobile edge computing. In another embodiment, the external electronic device 104 may include an Internet-of-things (IoT) device. The server 108 may be an intelligent server using machine learning and/or a neural network. According to an embodiment, the external electronic device 104 or the server 108 may be included in the second network 199. The electronic device 101 may be applied to intelligent services (e.g., smart home, smart city, smart car, or health-care) based on 5G communication technology or IoT-related technology.

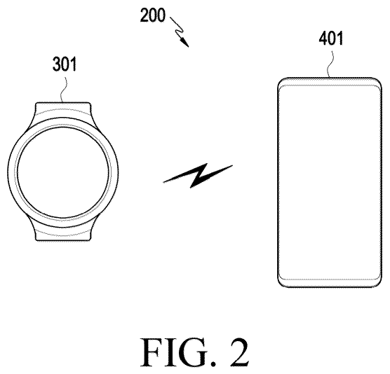

FIG. 2 is a view 200 illustrating a system capable of determining a user's activity state using a plurality of electronic devices according to an embodiment of the disclosure.

Referring to FIG. 2, a system capable of determining the activity state of the user may include a first electronic device 301 and a second electronic device 401.

According to an embodiment, the first electronic device 301 (e.g., the electronic device 101 of FIG. 1) may detect a first activity state indicating the user's activity state in the first electronic device 301 and, upon receiving a second activity state corresponding to a result of identifying the first activity state in a second electronic device 401 from the second electronic device 401 (e.g., the electronic device 102 or the electronic device 104 of FIG. 1) in response to a request for identifying the first activity state, update the user's activity state based on the second activity state.

According to an embodiment, the first electronic device 301 (e.g., the electronic device 101 of FIG. 1) may detect a first activity state indicating the user's activity state in the first electronic device 301 and, upon receiving a second activity state indicating the user's activity state detected by the second electronic device 401 from the second electronic device in response to a request for the user's activity state detected by the second electronic device 401, update the user's activity state based on the second activity state.

According to an embodiment, the first electronic device 301 may include a wearable electronic device and may include, e.g., a smart watch.

According to an embodiment, the first activity state may include one of various activity states of the user, which may be detected based on at least one of a sensor module (e.g., the sensor module 176 of FIG. 1) or a communication module (e.g., the communication module 190 of FIG. 1) included in the first electronic device 301, and may include, e.g., a walking state, a biking state, and/or a vehicle boarding state.

According to an embodiment, when the second activity state (e.g., the biking state) received from the second electronic device 401 is the same as the first activity state (e.g., the biking state) detected by the first electronic device 301, the first electronic device 301 may update the first activity state (e.g., the biking state) to the user's activity state.

According to an embodiment, when the second activity state (e.g., the walking state) received from the second electronic device 401 is different from the first activity state (e.g., the biking state) detected by the first electronic device 301, the first electronic device 301 may update the second activity state (e.g., the walking state) to the user's activity state.

According to an embodiment, upon receiving the request for identifying the first activity state detected by the first electronic device 301 from the first electronic device 301, the second electronic device 401 (e.g., the electronic device 102 or the electronic device 104 of FIG. 1) may detect the second activity state corresponding to the result of identifying the first activity state and may transmit the second activity state to the first electronic device 301. According to an embodiment, the second electronic device 401 may include identical or similar components to the electronic device 101 of FIG. 1.

According to an embodiment, if the second electronic device 401 (e.g., the electronic device 102 or the electronic device 104 of FIG. 1) receives the request for the user's activity state detected by the second electronic device 401 from the first electronic device 301, the second electronic device 401 may detect the second activity state indicating the user's activity state and may transmit the second activity state to the first electronic device 301.

According to an embodiment, the second activity state may include one of various activity states of the user, and may include, e.g., a walking state, a biking state, and/or a vehicle boarding state.

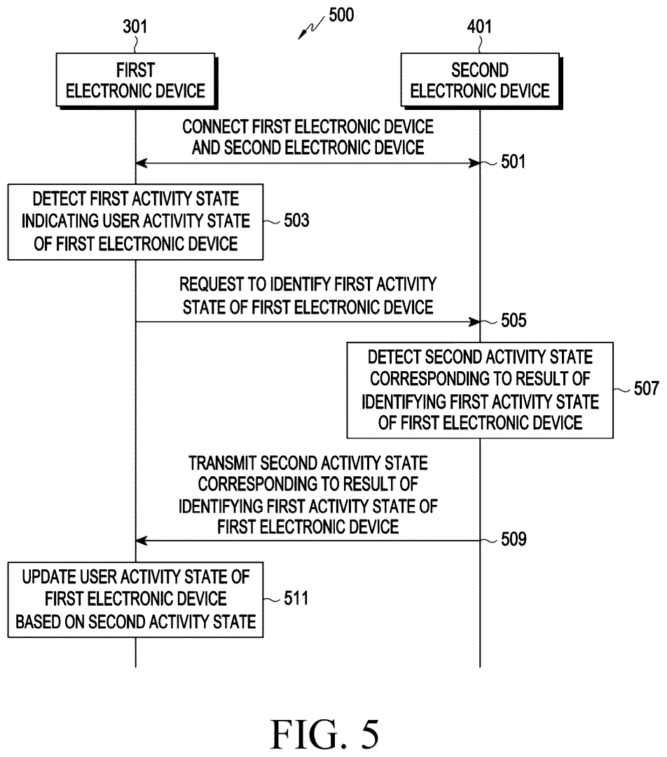

According to an embodiment, the second electronic device 401 may identify occurrence of a walking step using at least one of a sensor module (e.g., the second sensor module 476 of FIG. 4) or a communication module (e.g., the second communication module 490 of FIG. 4) of the second electronic device, and may detect the walking state as the second activity state when it is identified that the second electronic device is carried by the user. For example, if biometric information (e.g., an electrical signal) about the user is obtained using the sensor module of the second electronic device, the second electronic device 401 may identify that the second electronic device is carried in the user's hand. For example, the second electronic device 401 may identify that the second electronic device is carried in the user's hand using a grip sensor among sensor modules of the second electronic device. For example, the second electronic device 401 may identify that the second electronic device is carried in a garment (e.g., a pocket of an outer garment) and/or an accessory (e.g., a bag) worn by the user using an inertia sensor among sensor modules of the second electronic device.

According to an embodiment, upon identifying that a walking step occurs and that the second electronic device is carried by the user using at least one of the sensor module or the communication module of the second electronic device, and identifying that the moving speed of the second electronic device is less than a first threshold speed (e.g., about 10 km/h), the second electronic device 401 may detect the walking state as the second activity state.

According to an embodiment, upon identifying that the second electronic device is located outdoors using at least one of the sensor module or the communication module of the second electronic device and identifying that the moving speed of the second electronic device meets a threshold speed range which is a range of the first threshold speed (e.g., about 10 km/h) or more and a second threshold speed (e.g., about 25 km/h) or less, the second electronic device 401 may detect the biking state as the second activity state.

According to an embodiment, the second electronic device 401 may detect at least one of vibration pattern information about the vehicle, magnetic pattern information corresponding to the traveling state of the vehicle, or engine output sound information about the vehicle using at least one of the sensor module or the communication module of the second electronic device, and upon identifying that the moving speed of the second electronic device exceeds the second threshold speed (e.g., about 25 km/h), the second electronic device 401 may detect the vehicle boarding state as the second activity state.

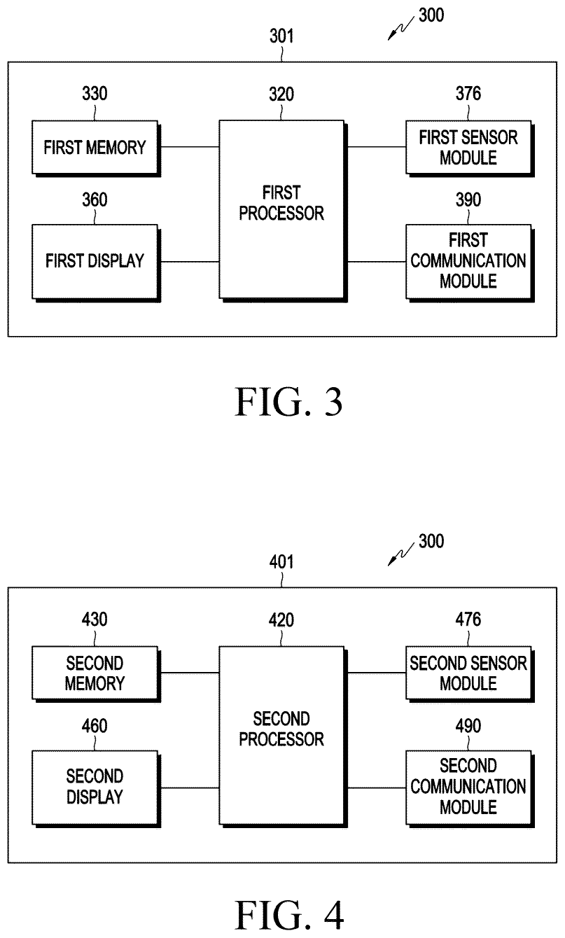

FIG. 3 is a block diagram 300 illustrating a first electronic device according to an embodiment of the disclosure.

Referring to FIG. 3, according to an embodiment, the first electronic device 301 may include a first processor 320, a first sensor module 376, a first communication module 390, first memory 330, and a first display 360.

At least some of the components of the electronic device 301 illustrated in FIG. 3 may be identical or similar to the components of the electronic device 101 of FIG. 1 and/or the first electronic device 301 illustrated in FIG. 2, and no duplicate description is given below.

According to an embodiment, the first processor 320 may be implemented to be substantially identical or similar to the processor 120 of FIG. 1.

According to an embodiment, the first processor 320 may detect the first activity state indicating the user's activity state, based on at least one of the first sensor module 376 or the first communication module 390.

According to an embodiment, the processor 320 may detect the first activity state indicating the user's activity state, based on at least one sensor (e.g., an acceleration sensor and/or a gyro sensor) among the plurality of sensors included in the first sensor module 376 or at least one communication module (e.g., a GPS communication module) among the plurality of communication modules included in the first communication module 390.

For example, when the first processor 320 detects a designated walking pattern based on sensor data obtained through the acceleration sensor of the first sensor module 376, the first processor 320 may determine that the current activity state of the user is the walking state, and may detect the walking state as the first activity state.

For example, upon detecting the vibration pattern of the road surface corresponding to biking based on sensor data obtained through the acceleration sensor of the first sensor module 376, and detecting the bicycle handling motion based on sensor data obtained through the gyro sensor of the first sensor module 376, the processor 320 may determine the current activity state of the user as the biking state, and detect the biking state as the first activity state.

For example, upon detecting the vibration pattern of the road surface corresponding to vehicle boarding based on sensor data obtained through an acceleration sensor of the first sensor module 376, and detecting the vehicle handling motion based on sensor data obtained through a gyro sensor of the first sensor module 376, the processor 320 may determine the current user activity state as the vehicle boarding state, and detect the vehicle boarding state as the first activity state. Additionally, the first processor 320 may identify whether the first activity state of the first electronic device is the vehicle boarding state based on the moving speed of the first electronic device detected using the GPS communication module of the first communication module 390, but the GPS communication module may be used considering the battery capacity of the first electronic device and/or a specific condition.

According to an embodiment, in order to identify the first activity state detected by the first electronic device 301, the first processor 320 may request a second electronic device (e.g., the electronic device 102, the electronic device 104, and/or the second electronic device 401 of FIG. 1) to identify the first activity state, and upon receiving a second activity state corresponding to the result of identifying the first activity state from the second electronic device, update the user's activity state based on the second activity state.

According to an embodiment, the first processor 320 may be connected to the second electronic device through the first communication module 390. According to an embodiment, the first communication module 390 may include at least one of a wireless LAN module and a short-range communication module, and may include an ultra-wide band (UWB) communication module, a Wi-Fi communication module, an NFC communication module, a Bluetooth legacy communication module, and/or a BLE communication module as the short-range communication module (not illustrated).

According to an embodiment, the first processor 320 may request the second electronic device to identify the first activity state through the first communication module 390.

According to an embodiment, upon receiving the second activity state corresponding to the result of identifying the first activity state from the second electronic device through the first communication module 390, the first processor 320 may update the user's activity state based on whether the first activity state detected by the first electronic device is the same as the second activity state detected by the second electronic device.

According to an embodiment, if the second activity state (e.g., the biking state) received from the second electronic device is the same as the first activity state (e.g., the biking state) detected by the first electronic device 301, the first processor 320 may update the first activity state (e.g., the biking state) to the user's activity state.

According to an embodiment, if the second activity state (e.g., the vehicle boarding state) received from the second electronic device is different from the first activity state (e.g., the bicycle boarding state) detected by the first electronic device 301, the first processor 320 may update the second activity state (e.g., the vehicle boarding state) to the user's activity state.

For example, if the user activity state of the first electronic device is updated to the walking state based on the second activity state (e.g., the walking state) of the second electronic device, the first processor 320 may operate a fall detection algorithm used in the walking state. The first processor 320 may receive the recognized walk information from the second electronic device and update the walk information in the first electronic device based on the received walk information.

For example, if the user activity state of the first electronic device is updated to the biking state based on the second activity state (e.g., the biking state) of the second electronic device, the first processor 320 may operate the fall detection algorithm used in the biking state.

For example, if the user activity state of the first electronic device is updated to the vehicle boarding state based on the second activity state (e.g., the vehicle boarding state) of the second electronic device, the first processor 320 may operate a vehicle collision accident detection algorithm.

According to an embodiment, after updating the user activity state of the first electronic device based on the second activity state received from the second electronic device, the first processor 320 may transmit a message requesting synchronization of the user activity state of the second electronic device to the user activity state of the first electronic device to the second electronic device.

According to an embodiment, in order to identify the first activity state detected by the first electronic device 301, the first processor 320 may request the second electronic device (e.g., the electronic device 102, the electronic device 104, and/or the second electronic device 401 of FIG. 1) to identify the user's activity state detected by the second electronic device 401, and upon receiving the second activity state indicating the user's activity state detected by the second electronic device from the second electronic device, update the user's activity state based on the second activity state.

According to an embodiment, the first processor 320 may request the second activity state (e.g., the walking state, the biking state, or the vehicle boarding state) indicating the user activity state currently detected by the second electronic device through the first communication module 390.

According to an embodiment, the first processor 320 may update the user activity state of the first electronic device, based on the second activity state (e.g., the walking state, the biking state, or the vehicle boarding state) indicating the user activity state of the second electronic device received through the first communication module 390.

According to an embodiment, after updating the user activity state of the first electronic device, the first processor 320 may transmit a message requesting to update the user activity state of the second electronic device to the user activity state of the first electronic device to the second electronic device in order to synchronize the first electronic device and the second electronic device.

According to an embodiment, in order to identify the first activity state detected by the first electronic device 301, the first processor 320 may request information for determining the second activity state indicating the user activity state of the second electronic device 401 from the second electronic device (e.g., the electronic device 102, the electronic device 104, and/or the second electronic device 401 of FIG. 1), determine the activity state of the second electronic device based on the information for determining the second activity state received from the second electronic device, and determine the user's activity state based on the determined second activity state.

According to an embodiment, the first processor 320 may request, from the second electronic device, at least one of information for determining the second activity state of the second electronic device, e.g., information for determining the walking state, information for determining the biking state, or information for determining the vehicle boarding state.

For example, the first processor 320 may request information (e.g., walk occurrence information and carry information indicating whether the second electronic device is carried by the user) for determining the walking state from the second electronic device, and, upon determining the activity state corresponding to the user activity state of the second electronic device as the walking state based on the information for determining the walking state received from the second electronic device, update the user activity state of the first electronic device to the walking state.

For example, the first processor 320 may request information (e.g., information indicating that the second electronic device is located outdoors and moving speed information about the second electronic device) for determining the biking state from the second electronic device, and if it is determined that the activity state corresponding to the user activity state of the second electronic device is the biking state based on the information for determining the biking state received from the second electronic device, the first processor 320 may update the user activity state of the first electronic device to the biking state.

For example, the first processor 320 may request information (e.g., at least one of vibration pattern information about the vehicle, magnetic pattern information corresponding to the traveling state of the vehicle, or engine output sound information about the vehicle and moving speed information of the second electronic device) for determining the vehicle boarding state from the second electronic device, and if it is determined that the activity state corresponding to the user activity state of the second electronic device is the vehicle boarding state based on the information for determining the vehicle boarding state received from the second electronic device, the first processor 320 may update the user activity state of the first electronic device to the vehicle boarding state.

For example, after detecting the first activity state of the first electronic device as the biking state, the first processor 320 may request information for determining that the second activity state of the second electronic device is the walking state from the second electronic device. if it is determined that the second activity state of the second electronic device is the walking state based on the information for determining the walking state received from the second electronic device, the first processor 320 may update the user activity state of the first electronic device to the walking state. Upon identifying that the second activity state of the second electronic device is not the walking state based on the information for determining the walking state received from the second electronic device, the first processor 320 may request information for determining that the second activity state of the second electronic device is the bicycle state from the second electronic device. Upon identifying that the second activity state of the second electronic device is not the walking state based on the information for determining the walking state received from the second electronic device, the first processor 320 may request information for determining that the second activity state of the second electronic device is the bicycle state from the second electronic device.

According to an embodiment, the first sensor module 376 may be implemented in the same or similar manner to the sensor module 176 of FIG. 1.

According to an embodiment, the sensor module 376 may include an acceleration sensor, a gyro sensor, or the like.

According to an embodiment, the first sensor module 376 may transmit, to the first processor 320, sensor data measured to detect the first activity state (e.g., the walking state, the biking state, or the vehicle boarding state) indicating the user's activity state in the first electronic device 301.

According to an embodiment, the first communication module 390 may be implemented to be substantially identical or similar to the communication module 190 of FIG. 1 and may include a plurality of communication circuits using different communication technologies.

According to an embodiment, the first communication module 390 may include at least one of a wireless LAN module and a short-range communication module, and may include an ultra-wide band (UWB) communication module, a Wi-Fi communication module, an NFC communication module, a Bluetooth legacy communication module, and/or a BLE communication module as the short-range communication module.

According to an embodiment, the first communication module 390 may include a GPS communication module as the wireless LAN module, and may detect the moving speed of the first electronic device 301 through the GPS communication module.

According to an embodiment, the first memory 330 may be implemented to be substantially identical or similar to the memory 130 of FIG. 1.

According to an embodiment, the first display 360 may be implemented in substantially the same or similar manner to the display module 160 of FIG. 1.

According to an embodiment, the first display 360 may display the image and/or information indicating the type of the first activity state (e.g., the walking state, the biking state, or the vehicle boarding state) indicating the user activity state of the first electronic device.

According to an embodiment, the first display 360 may display information (e.g., walking step information, bicycle exercise information, or vehicle speed information) that is changed according to the movement of the first electronic device in the first activity state.

FIG. 4 is a block diagram 400 illustrating a second electronic device according to an embodiment of the disclosure.

Referring to FIG. 4, according to an embodiment, the second electronic device 401 may include a second processor 420, a second sensor module 476, a second communication module 490, second memory 430, and a second display 460.

At least some of the components of the electronic device 401 illustrated in FIG. 4 may be identical or similar to the components of the electronic device 102 or electronic device 104 of FIG. 1 and/or the second electronic device 401 illustrated in FIG. 2, and no duplicate description is given below.

According to an embodiment, upon receiving the request for identifying the first activity state detected by the first electronic device from the first electronic device (e.g., the first electronic device 301 of FIG. 2 and/or the first electronic device 301 of FIG. 3), the second electronic device 420 (e.g., the electronic device 102 or the electronic device 301 of FIG. 1) may detect the second activity state corresponding to the result of identifying the first activity state and may transmit the second activity state to the first electronic device 301 through the second communication module 490.

According to an embodiment, the second processor 420 may be connected to the first electronic device through the second communication module 490. According to an embodiment, the second communication module 490 may include at least one of a wireless LAN module and a short-range communication module, and may include an ultra-wide band (UWB) communication module, a Wi-Fi communication module, an NFC communication module, a Bluetooth legacy communication module, and/or a BLE communication module as the short-range communication module (not illustrated).

According to an embodiment, the second activity state may include one of various activity states of the user, which may be detected based on at least one of the second sensor module 476 or the second communication module 490, and may include, e.g., a walking state, a biking state, or a vehicle boarding state.

According to an embodiment, upon receiving the request for identifying the first activity state detected by the first electronic device 301 from the first electronic device through the second communication module 490, the second processor 420 may detect the second activity state corresponding to the result of identifying the first activity state based on at least one of the second sensor module 476 or the second communication module 490. The second activity state may include at least one of information indicating whether the second activity state is the same as the first activity state or information indicating the type of the second activity state (e.g., the walking state, biking information, or vehicle boarding information).

According to an embodiment, upon receiving the request for identifying the first activity state (e.g., the bicycle boarding state) detected by the first electronic device 301, the second processor 420 may detect a single vector magnitude (SVM) signal from the sensor data obtained from the acceleration sensor of the second sensor module 476, and may determine whether a walking step occurs by checking whether a designated walking pattern occurs in the detected signal. The single vector magnitude (SVM) signal is a magnitude value of three axes of acceleration and may be calculated through Equation 1 below.

$$SVM = \sqrt{{x}^{2} + {y}^{2} + {z}^{2}} \qquad [ Equation 1 ]$$

Equation 1 above is merely an example for helping understanding, and the method for calculating the SVM signal is not limited thereto, may be modified, applied, or expanded in various ways.

The second processor 420 may identify the occurrence of a walking step by identifying the occurrence of the designated walking pattern, and upon identifying that the second electronic device 401 is carried by the user based on sensor data obtained from the second sensor module 476, detect the walking state as the second activity state.

For example, if biometric information (e.g., an electrical signal) about the user is obtained using the sensor module 476, the second electronic device 420 may identify that the second electronic device 401 is carried in the user's hand.

For example, the second processor 420 may identify that the second electronic device 401 is carried in the user's hand using a grip sensor among sensor modules 476.

For example, the second processor 420 may identify that the second electronic device 401 is carried in a garment (e.g., a pocket of an outer garment) and/or an accessory (e.g., a bag) worn by the user using an inertia sensor among sensor modules 476.

According to an embodiment, the second processor 420 may identify the designated walking pattern in the second electronic device 401, upon identifying that the second electronic device 401 is not carried by the user, identify whether the second electronic device 401 is located indoors or outdoors, and upon identifying that the moving speed of the second electronic device 401 is less than a first threshold speed (e.g., about 10 km/h), detect the walking state as the second activity state.

According to an embodiment, the second processor 420 may identify whether the second electronic device 401 is located outdoors or indoors using the GPS communication module and/or the WI-FI communication module of the second communication module 490. For example, if the accuracy (e.g., a GPS accuracy value indicating an error range) of the location information measured through the GPS communication module of the second communication module 490 is a predetermined threshold or less (e.g., about 10 m or less, or about 20 m or less), or the number of satellites caught by the GPS communication module is a predetermined number or more (e.g., three or more), the second processor 420 may determine that the second electronic device 401 is located outdoors. For example, if the RSSI signal of the Wi-Fi communication module of the second communication module 490 is a predetermined threshold or less (e.g., about −70 dBm or less), the second processor 420 may determine that the second electronic device 401 is located outdoors.

According to an embodiment, when receiving a request for identifying the first activity state (e.g., the bicycle boarding state) detected by the first electronic device 301, if the occurrence of the user's walking step is not identified by the second electronic device 401, the second processor 420 may identify that the second electronic device 401 is located outdoors using at least one of the second sensor module 476 or second communication module 490 of the second electronic device and, upon identifying that the moving speed of the second electronic device meets a range of a first threshold speed (e.g., about 10 km/h) or more and a second threshold speed (e.g., about 25 km/h) or less which is a threshold speed range, detect the biking state as the second activity state.

For example, if the occurrence of the user's walking step is not identified by the second electronic device 401, the moving speed of the second electronic device 401 meets the range of the first threshold speed (e.g., about 10 km/h) or more and the second threshold speed (e.g., about 25 km/h) or less which is the threshold speed range, determine the second activity state corresponding to the user activity state of the second electronic device 401 as the biking state in which the user rides the bicycle with the second electronic device 401 mounted on the bicycle.

For example, if the occurrence of the user's walking step is not identified by the second electronic device 401, and the moving speed of the second electronic device 401 is less than the first threshold speed, the second processor 420 may determine the second activity state corresponding to the user activity state of the second electronic device 401 as the walking state in which a cart or stroller is pulled with the second electronic device 401 mounted on, e.g., a cup holder of the cart or stroller. According to an embodiment, if the accuracy (e.g., a GPS accuracy value indicating an error range) of the location information measured through the GPS communication module of the second communication module 490 is a predetermined threshold or less (e.g., about 10 m or less, or about 20 m or less), or the number of satellites caught by the GPS communication module is a predetermined number or more (e.g., three or more), or the RSSI signal of the Wi-Fi communication module of the second communication module 490 is a predetermined threshold or less (e.g., about-70 dBm or less), the second processor 420 may determine that the second electronic device 401 is located outdoors.

According to an embodiment, the second processor 420 may determine the moving speed of the second electronic device through the GPS communication module of the second communication module 490 and, if it is included in a threshold speed range (e.g., a bikeable speed), detect the biking state as the second activity state.

According to an embodiment, when the occurrence of the user's walking step is not identified by the second electronic device 401, the second processor 420 may identify whether the second electronic device 401 is located outdoors or indoors using the second communication module 490 and, upon identifying that the moving speed of the second electronic device 401 is less than the first threshold speed (e.g., about 10 km/h), detect the walking state as the second activity state.

According to an embodiment, the second processor 420 may detect at least one of vibration pattern information about the vehicle, magnetic pattern information corresponding to the traveling state of the vehicle, or engine output sound information about the vehicle using at least one of the second sensor module 476 or the second communication module 490 when receiving the request for identifying the first activity state (e.g., the bicycle boarding state) detected by the first electronic device 301, and upon identifying that the moving speed of the second electronic device exceeds the second threshold speed, may detect the vehicle boarding state as the second activity state.

According to an embodiment, the second processor 420 may detect the vibration pattern information about the vehicle using the acceleration sensor of the second sensor module, detect geomagnetic pattern information indicating the traveling state of the vehicle using the geomagnetic sensor of the second sensor module, and detect the engine output sound information or low-frequency signal of the vehicle through a sound sensor (e.g., a microphone) (not shown).

According to an embodiment, the second processor 420 may transmit, to the first electronic device through the second communication module 490, the second activity state (e.g., the walking state, biking state, or vehicle boarding state) indicating the user's activity state detected by the second electronic device 401, as the result of identifying the first activity state.

According to an embodiment, the second processor 420 may update the bicycle exercise information based on the bicycle exercise information received from the first electronic device in a state in which the user activity state of the second electronic device is synchronized to the user activity state (e.g., the bicycle boarding state) of the first electronic device.

According to an embodiment, upon receiving the request for the user activity state detected by the second electronic device 401 from the first electronic device (e.g., the first electronic device 301 of FIG. 2 and/or the first electronic device 301 of FIG. 3), the second processor 420 may detect the second activity state indicating the user activity state in the second electronic device 401 and transmit the second activity state to the first electronic device 301 through the second communication module 490.

According to an embodiment, upon receiving the request for the user's activity state detected by the second electronic device 401 from the first electronic device through the second communication module 490, the second processor 420 may detect the second activity state (e.g., the walking state, biking state, or vehicle boarding state) indicating the user activity state currently detected by the second electronic device 401 and transmit the detected second activity state to the first electronic device through the second communication module 490.

According to an embodiment, upon receiving the request for information for determining the second activity state indicating the user activity state of the second electronic device 401 from the first electronic device (e.g., the first electronic device 301 of FIG. 2 and/or the first electronic device 301 of FIG. 3), the second processor 420 may detect the information for determining the second activity state indicating the user's activity state in the second electronic device 401 and transmit the detected second activity state to the first electronic device 301 through the second communication module 490.

According to an embodiment, upon receiving the request for the information for determining the second activity state of the second electronic device, e.g., at least one of information for determining the walking state, information for determining the biking state, or information for determining the vehicle boarding state, from the first electronic device, the second processor 420 may detect the information for determining the second activity state and transmit it to the first electronic device 301 through the second communication module 490.

For example, upon receiving for the information for determining the walking state (e.g., walking step occurrence information and carry information where the second electronic device is carried by the user) from the first electronic device, the second processor 420 may detect the information for determining the walking state and transmit it to the first electronic device through the second communication module 490.

For example, upon receiving the request for the information for determining the biking state (e.g., information indicating that the second electronic device is located outdoors and moving speed information about the second electronic device) from the first electronic device, the second processor 420 may detect the information for determining the biking state and transmit it to the first electronic device through the second communication module 490.

For example, upon receiving the request for the information for determining the vehicle boarding state (at least one of vibration pattern information about the vehicle, magnetic pattern information corresponding to the traveling state of the vehicle, or engine output sound information about the vehicle and moving speed information about the second electronic device) from the first electronic device, the second processor 420 may detect the information for determining the vehicle boarding state and transmit it to the first electronic device through the second communication module 490.

According to an embodiment, the first sensor module 476 may be implemented in the same or similar manner to the sensor module 176 of FIG. 2.

According to an embodiment, the second sensor module 476 may include an acceleration sensor, a gyro sensor, a geomagnetic sensor, and/or a sound sensor.

According to an embodiment, the second sensor module 476 may transmit, to the second processor 420, sensor data measured to detect the first activity state (e.g., the walking state, the biking state, or the vehicle boarding state) indicating the user's activity state in the second electronic device 401.

According to an embodiment, the second communication module 490 may be implemented to be substantially identical or similar to the communication module 190 of FIG. 1 and may include a plurality of communication circuits using different communication technologies.

According to an embodiment, the second communication module 490 may include at least one of a wireless LAN module and a short-range communication module, and may include an ultra-wide band (UWB) communication module, a Wi-Fi communication module, an NFC communication module, a Bluetooth legacy communication module, and/or a BLE communication module as the short-range communication module.

According to an embodiment, the second communication module 490 may include a GPS communication module as the wireless LAN module, and may detect the moving speed of the second electronic device 401 through the GPS communication module.

According to an embodiment, the first memory 430 may be implemented to be substantially identical or similar to the memory 130 of FIG. 2.

According to an embodiment, the first display 460 may be implemented in substantially the same or similar manner to the display module 160 of FIG. 2.

According to an embodiment, the second display 460 may display the image and/or information indicating the type of the second activity state (e.g., the walking state, the biking state, or the vehicle boarding state) indicating the user activity state of the second electronic device.

According to an embodiment, the second display 460 may display information changed according to the movement of the second electronic device (e.g., walking step information, bicycle exercise information, and/or vehicle speed information) in the second activity state or information changed according to the movement of the first electronic device (e.g., walking step information, bicycle exercise information, and/or vehicle speed information) received from the first electronic device 301.

According to an embodiment, a system for determining a user's activity state, according to embodiments of the disclosure, may include a first electronic device (e.g., the first electronic device 301 of FIG. 2 and/or the first electronic device of FIG. 3) configured to request a second electronic device to identify a first activity state detected as a user's activity state in the first electronic device, receive a second activity state corresponding to a result of identifying the first activity state in the second electronic device received from the second electronic device in response to the request and update the user's activity state based on second activity state, and the second electronic device (e.g., the second electronic device 401 of FIG. 2 and/or the second electronic device 401 of FIG. 4) configured to, upon receiving the request for identifying the first activity state from the first electronic device, identify whether the user's activity state is the first activity state and transmit the second activity state corresponding to the identification result to the first electronic device.

According to an embodiment, the first electronic device may include a wearable electronic device.

According to an embodiment, the first electronic device may be configured to detect the first activity state based on at least one of a sensor module or a communication module included in the first electronic device.

According to an embodiment, the first electronic device may be configured to update the first activity state as the user's activity state if the second activity state received from the second electronic device is identical to the first activity state.

According to an embodiment, the first electronic device may be configured to update the second activity state to the user's activity state if the second activity state received from the second electronic device is different from the first activity state.

According to an embodiment, the second electronic device 401 may be configured to, upon identifying that a walking step occurs, and the second electronic device is carried by the user using a sensor module of the second electronic device, detect the user's walking state as the second activity state, upon identifying that the second electronic device is located indoor, and a moving speed of the second electronic device meets a threshold speed range using at least one of the sensor module or communication module of the second electronic device, detect a biking state as the second activity state, and detect at least one vibration pattern information about the vehicle, vehicle engine output sound information, or magnetic pattern information corresponding to a traveling state of the vehicle using at least one of the sensor module or the communication module of the second electronic device and, upon identifying that the moving speed of the second electronic device exceeds a second threshold speed, detect a vehicle boarding state as the second activity state.

According to an embodiment, an electronic device (e.g., the first electronic device 301 of FIG. 2 and/or the first electronic device 301 of FIG. 3) may include a sensor module (e.g., the first sensor module 376 of FIG. 3), a communication module (e.g., the first communication module 390 of FIG. 3), and at least one processor (e.g., the first processor 320 of FIG. 3). The processor may be configured to detect a first activity state indicating a user activity state based on at least one of the sensor module or the communication module, receive a second activity state corresponding to a result of identifying the first activity state in the first electronic device from the first electronic device (e.g., the second electronic device 401 of FIG. 2 and/or the second electronic device 401 of FIG. 4) through the communication module in response to the request for identifying the first activity state, and update the user activity state based on the second activity state received from the first electronic device (e.g., the second electronic device 401 of FIG. 2 and/or the second electronic device 401 of FIG. 4).

According to an embodiment, the processor may be configured to update the first activity state to the user's activity state if the second activity state received from the first electronic device is identical to the first activity state and update the second activity state to the user's activity state if the second activity state received from the first electronic device is different from the first activity state.

According to an embodiment, an electronic device (e.g., the second electronic device 401 of FIG. 2 and/or the second electronic device 401 of FIG. 4) may include a communication module (e.g., the second communication module 476 of FIG. 4), a sensor module (e.g., the second sensor module 490 of FIG. 4), and at least one processor (e.g., the second processor 420 of FIG. 4). The processor may be configured to, upon receiving a request for identifying the first activity state from a first electronic device (e.g., the first electronic device 301 of FIG. 2 and/or the first electronic device 301 of FIG. 3), detect a second activity state corresponding to a result of identifying whether a user activity state is the first activity state based on at least one of the sensor module or the communication module, and transmit the second activity state to the first electronic device through the communication module.

According to an embodiment, the processor may be configured to, upon identifying that a walking step occurs, and the second electronic device is carried by the user using a sensor module, detect the user's walking state as the second activity state, upon identifying that the second electronic device is located indoor, and a moving speed of the second electronic device meets a threshold speed range using at least one of the sensor module or communication module, detect a biking state as the second activity state, and detect at least one of magnetic pattern information or vehicle engine output sound information corresponding to a traveling state of the vehicle and vibration pattern information about the vehicle using at least one of the sensor module or communication module and, upon identifying that the moving speed of the second electronic device exceeds a second threshold speed, detect a vehicle boarding state as the second activity state.

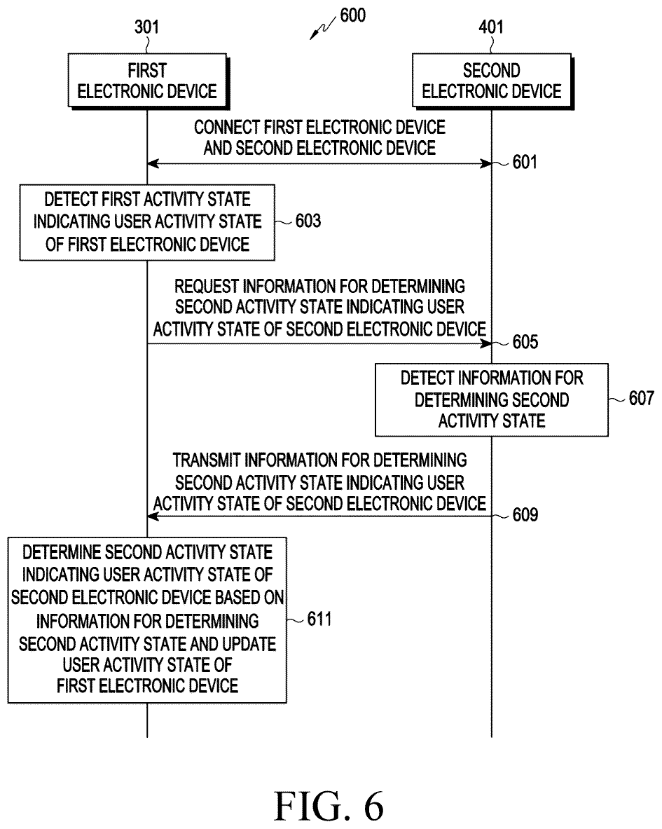

FIG. 5 is a flowchart 500 illustrating an operation of determining a user's activity state using a plurality of electronic devices according to an embodiment of the disclosure. The operations of determining the user's activity state may include operations 501 to 511. In some embodiments, each operation may be performed sequentially, but is not necessarily required to be performed sequentially. For example, the order of the operations may be changed, and at least two operations may be performed in parallel or other operations may be added.

In an embodiment, operations 501 to 511 may be understood as performed by the processor (e.g., the first processor 320 of FIG. 3 or second processor 420 of FIG. 4) of each electronic device (e.g., the first electronic device 301 or the second electronic device 401).

In operation 501, the first electronic device 301 (e.g., the first electronic device 301 of FIG. 2 and/or the first electronic device 301 of FIG. 3) and the second electronic device 401 (e.g., the second electronic device 401 of FIG. 2 and/or the second electronic device 401 of FIG. 4) may be connected to each other through the first communication module (e.g., the first communication module 390 of FIG. 3) and the second communication module (e.g., the second communication module 490 of FIG. 4).

According to an embodiment, they may be connected through short-range communication using the communication module (e.g., the first communication module 390 of FIG. 3) of the first electronic device and the communication module (e.g., the second communication module 490 of FIG. 4) of the second electronic device. The short-range communication may mean all communication schemes capable of, e.g., a time difference of arrival (TDoA) scheme, as the same wireless communication scheme without specifying the specifications of the first electronic device 301 and the second electronic device 401. An example may include, but is not limited to, Bluetooth communication, Bluetooth low energy (BLE) communication, Wi-Fi communication, near-field communication (NFC) communication, and/or ultra-wideband (UWB) communication.

In operation 503, the first electronic device 301 (e.g., the first electronic device 301 of FIG. 2 and/or the first electronic device 301 of FIG. 3) may detect the first activity state indicating the user activity state of the first electronic device.

According to an embodiment, the first electronic device 301 may detect the first activity state indicating the user activity state of the first electronic device, based on at least one sensor (e.g., an acceleration sensor and/or a gyro sensor) among the plurality of sensors included in the sensor module (e.g., the first sensor module 376 of FIG. 3) of the first electronic device or at least one communication module (e.g., a GPS communication module) among the plurality of communication modules included in the communication module (e.g., the first communication module 390 of FIG. 3) of the first electronic device.

For example, upon detecting a designated walking pattern based on sensor data obtained through the acceleration sensor included in the sensor module of the first electronic device, the first electronic device 301 may determine that the current activity state of the user is the walking state, and may detect the walking state as the first activity state.

For example, upon detecting the vibration pattern of the road surface corresponding to biking based on sensor data obtained through the acceleration sensor included in the sensor module of the first electronic device, and detecting the bicycle handling motion based on sensor data obtained through the gyro sensor included in the sensor module of the first electronic device, the first electronic device 301 may determine the current activity state of the user as the biking state, and detect the biking state as the first activity state.

For example, upon detecting the vibration pattern of the road surface corresponding to vehicle boarding based on sensor data obtained through an acceleration sensor included in the sensor module of the first electronic device, and detecting the vehicle handling motion based on sensor data obtained through a gyro sensor included in the sensor module of the first electronic device, the first electronic device 301 may determine the current user activity state as the vehicle boarding state, and detect the vehicle boarding state as the first activity state. Additionally, the first electronic device may identify whether the first activity state of the first electronic device is the vehicle boarding state based on the moving speed of the first electronic device detected using the GPS communication module included in the communication module (e.g., the first communication module 390 of FIG. 3) of the first electronic device. The first electronic device may use the GPS communication module using the battery capacity of the first electronic device and/or a specific condition.

In operation 505, the first electronic device 301 (e.g., the first electronic device 301 of FIG. 2 and/or the first electronic device 301 of FIG. 3) may request to identify the first activity state of the first electronic device.

According to an embodiment, the first electronic device 301 may request the second electronic device to identify the first activity state through the communication module (e.g., the first communication module 390 of FIG. 3) of the first electronic device.

In operation 507, the second electronic device 401 (e.g., the second electronic device 401 of FIG. 2 and/or the second electronic device 401 of FIG. 4) may detect the second activity state corresponding to the result of identifying the first activity state of the first electronic device.

According to an embodiment, the second activity state may include one of various activity states of the user, which may be detected based on at least one of the sensor module (e.g., the second sensor module 476 of FIG. 4) of the second electronic device or the communication module (e.g., the second communication module 490 of FIG. 4) of the second electronic device 401, and may include, e.g., a walking state, a biking state, or a vehicle boarding state.

According to an embodiment, upon receiving the request for identifying the first activity state detected by the first electronic device 301 from the first electronic device 301 through the second communication module 490, the second electronic device 401 may detect the second activity state corresponding to the result of identifying the first activity state based on at least one of the second sensor module 476 or the second communication module 490.

In operation 509, the second electronic device 401 (e.g., the second electronic device 401 of FIG. 2 and/or the second electronic device 401 of FIG. 4) may transmit the second activity state corresponding to the result of identifying the first activity state of the first electronic device to the first electronic device 301.

According to an embodiment, the second electronic device 401 may transmit the detected second activity state to the first electronic device 301 through the communication module (e.g., the second communication module 490 of FIG. 4) of the second electronic device.

In operation 511, the first electronic device 301 (e.g., the first electronic device 301 of FIG. 2 and/or the first electronic device 301 of FIG. 3) may update the user activity state of the first electronic device based on the second activity state.

According to an embodiment, when the second activity state (e.g., the biking state) received from the second electronic device 401 is the same as the first activity state (e.g., the biking state) detected by the first electronic device 301, the first electronic device 301 may update the first activity state (e.g., the biking state) to the user's activity state. For example, upon updating the user activity state of the first electronic device to the biking state, the first electronic device 301 may operate a fall detection algorithm used in the biking state.

According to an embodiment, when the second activity state (e.g., the vehicle boarding state) received from the second electronic device 401 is different from the first activity state (e.g., the bicycle boarding state) detected by the first electronic device 301, the first electronic device 301 may update the second activity state (e.g., the vehicle boarding state) to the user's activity state. For example, upon updating the user activity state of the first electronic device to the vehicle boarding state, the first electronic device 301 may operate a vehicle collision accident detection algorithm.

According to an embodiment, as the first electronic device 301 and the second electronic device 401 are synchronized with the same activity state of the user, information collected based on the sensor data obtained through the sensor module included in each electronic device (e.g., the first electronic device 301 and the second electronic device 401) may be shared during the synchronized activity state of the user.

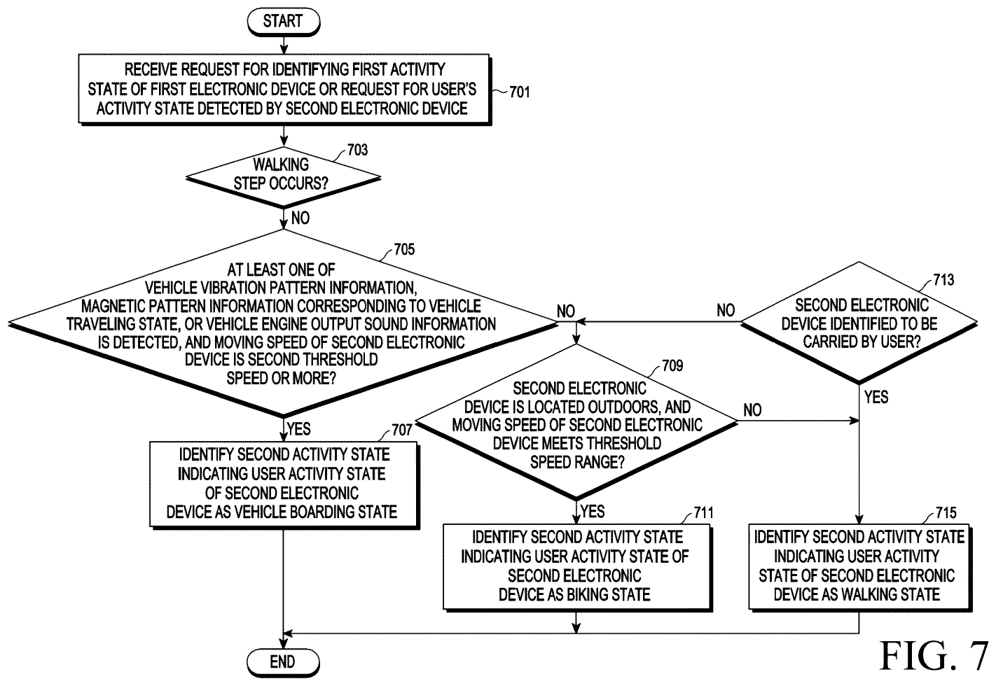

FIG. 6 is a flowchart 600 illustrating an operation of determining a user activity state using a plurality of electronic devices according to an embodiment of the disclosure. The operations of determining the user activity state may include operations 601 to 611. In some embodiments, each operation may be performed sequentially, but is not necessarily required to be performed sequentially. For example, the order of the operations may be changed, and at least two operations may be performed in parallel or other operations may be added.

According to an embodiment, operations 601 to 611 may be understood as performed by the processor (e.g., the first processor 320 of FIG. 3 or the second processor 420 of FIG. 4) of each electronic device (e.g., the first electronic device 301 or the second electronic device 401).

In operation 601, the first electronic device 301 (e.g., the first electronic device 301 of FIG. 2 and/or the first electronic device 301 of FIG. 3) and the second electronic device 401 (e.g., the second electronic device 401 of FIG. 2 and/or the second electronic device 401 of FIG. 4) may be connected to each other through the first communication module (e.g., the first communication module 390 of FIG. 3) and the second communication module (e.g., the second communication module 490 of FIG. 4).

According to an embodiment, the first electronic device 301 and the second electronic device 401 may be connected through short-range communication using the communication module (e.g., the first communication module 390 of FIG. 3) of the first electronic device and the communication module (e.g., the second communication module 490 of FIG. 4) of the second electronic device. The short-range communication may mean all communication schemes capable of, e.g., a time difference of arrival (TDoA) scheme, as the same wireless communication scheme without specifying the specifications of the first electronic device 301 and the second electronic device 401. An example may include, but is not limited to, Bluetooth communication, Bluetooth low energy (BLE) communication, Wi-Fi communication, near-field communication (NFC) communication, and/or ultra-wideband (UWB) communication.

In operation 603, the first electronic device 401 (e.g., the first electronic device 301 of FIG. 2 and/or the first electronic device 301 of FIG. 3) may detect the first activity state indicating the user activity state of the first electronic device.

According to an embodiment, the first electronic device 301 may detect the first activity state indicating the activity state of the user of the first electronic device based on at least one sensor (e.g., an acceleration sensor and/or a gyro sensor) among the plurality of sensors included in the sensor module (e.g., the first sensor module 376 of FIG. 3) of the first electronic device or at least one communication module (e.g., a GPS communication module) among the plurality of communication modules included in the communication module (e.g., the first communication module 390 of FIG. 3) of the first electronic device.

For example, upon detecting a designated walking pattern based on sensor data obtained through the acceleration sensor included in the sensor module of the first electronic device, the first electronic device 301 may determine that the current activity state of the user is the walking state, and may detect the walking state as the first activity state.

For example, upon detecting the vibration pattern of the road surface corresponding to biking based on sensor data obtained through the acceleration sensor included in the sensor module of the first electronic device, and detecting the bicycle handling motion based on sensor data obtained through the gyro sensor included in the sensor module of the first electronic device, the first electronic device 301 may determine the current activity state of the user as the biking state, and detect the biking state as the first activity state.

For example, upon detecting the vibration pattern of the road surface corresponding to vehicle boarding based on sensor data obtained through an acceleration sensor included in the sensor module of the first electronic device, and detecting the vehicle handling motion based on sensor data obtained through a gyro sensor included in the sensor module of the first electronic device, the first electronic device 301 may determine the current user activity state as the vehicle boarding state, and detect the vehicle boarding state as the first activity state. Additionally, the first electronic device may identify whether the first activity state of the first electronic device is the vehicle boarding state based on the moving speed of the first electronic device detected using the GPS communication module included in the communication module (e.g., the first communication module 390 of FIG. 3) of the first electronic device. The first electronic device may use the GPS communication module using the battery capacity of the first electronic device and/or a specific condition.

In operation 605, the first electronic device 301 (e.g., the first electronic device 301 of FIG. 2 and/or the first electronic device 301 of FIG. 3) may request the second activity state indicating the user activity state of the second electronic device 401 from the second electronic device (e.g., the second electronic device 401 of FIG. 2 and/or the second electronic device 401 of FIG. 4).

According to an embodiment, the first electronic device 301 may request, from the second electronic device 401, information for determining the second activity state indicating the user activity state of the second electronic device 401 through the communication module (e.g., the first communication module 390 of FIG. 3) of the first electronic device.

According to an embodiment, the first electronic device 301 may transmit, to the second electronic device 401, a request for information for determining the second activity state of the second electronic device 401, e.g., at least one of information for determining the walking state, information for determining the biking state, or information for determining the vehicle boarding state.

For example, the first electronic device 301 may transmit, to the second electronic device 401, a request for the information for determining the walking state (e.g., walking step occurrence information and carry information where it may be known whether the second electronic device is carried by the user).

For example, the first electronic device 301 may transmit, to the second electronic device 401, a request for the information for determining the biking state (e.g., information indicating that the second electronic device is located outdoors and moving speed information about the second electronic device 401).

For example, the first electronic device 301 may transmit, to the second electronic device 401, a request for the information for determining the vehicle boarding state (e.g., at least one of vibration pattern information about the vehicle, magnetic pattern information corresponding to the traveling state of the vehicle, or engine output sound information about the vehicle, and moving speed information about the second electronic device 401).

In operation 607, the second electronic device 401 (e.g., the second electronic device 401 of FIG. 2 and/or the second electronic device 401 of FIG. 4) may detect the information for determining the second activity state indicating the user activity state of the second electronic device 401.

According to an embodiment, the second activity state may include one of various activity states of the user, which may be detected based on at least one of the sensor module (e.g., the second sensor module 476 of FIG. 4) of the second electronic device or the communication module (e.g., the second communication module 490 of FIG. 4) of the second electronic device 401, and may include, e.g., a walking state, a biking state, or a vehicle boarding state.

According to an embodiment, the second electronic device 401 may detect the information for determining the second activity state indicating the user activity state of the second electronic device 401 based on at least one of the sensor module (e.g., the second sensor module 476 of FIG. 4) of the second electronic device or the communication module (e.g., the second communication module 490 of FIG. 4) of the second electronic device.

According to an embodiment, upon receiving the information for determining the second activity state of the second electronic device 401, e.g., at least one of the information for determining the walking state, the information for determining the biking state, or the information for determining the vehicle boarding state, as the request information for the user activity state detected by the second electronic device, from the first electronic device 301, the second electronic device 401 may detect the information for determining the second activity state.

For example, upon receiving the request for the information for determining the walking state (e.g., walking step occurrence information and carry information where it may be known whether the second electronic device is carried by the user) from the first electronic device 301, the second electronic device 401 may detect the information for determining the walking state and transmit it to the first electronic device 301.

For example, upon receiving the request for the information for determining the biking state (e.g., information indicating that the second electronic device is located outdoors and moving speed information about the second electronic device 401) from the first electronic device 301, the second electronic device 401 may detect the information for determining the biking state and transmit it to the first electronic device 301.

For example, upon receiving the request for the information for determining the vehicle boarding state (e.g., at least one of vibration pattern information about the vehicle, magnetic pattern information corresponding to the traveling state of the vehicle, or engine output sound information about the vehicle, and moving speed information about the second electronic device 401) from the first electronic device, the second electronic device 401 may detect the information for determining the vehicle boarding state and transmit it to the first electronic device 301.

In operation 609, the second electronic device 401 (e.g., the second electronic device 401 of FIG. 2 and/or the second electronic device 401 of FIG. 4) may transmit, to the first electronic device 301, the information for determining the second activity state indicating the user activity state of the second electronic device 401.

According to an embodiment, the second electronic device 401 may transmit the information for determining the second activity state indicating the user activity state of the second electronic device 401 to the first electronic device 301 through the communication module (e.g., the second communication module 490 of FIG. 4) of the second electronic device.

In operation 611, the first electronic device 301 (e.g., the first electronic device 301 of FIG. 2 and/or the first electronic device 301 of FIG. 3) may determine the second activity state indicating the user activity state of the second electronic device 401 based on the information for determining the second activity state and update the user activity state of the first electronic device based on the determined second activity state.

According to an embodiment, upon receiving the information for determining the second activity state of the second electronic device, e.g., at least one of the information for determining the walking state, the information for determining the biking state, or the information for determining the vehicle boarding state, the first electronic device 301 may determine the second activity state corresponding to the user activity state of the second electronic device based on the information for determining the second activity state and update the user activity state of the first electronic device based on the second activity state.

For example, upon receiving the request for the information for determining the walking state (e.g., walking step occurrence information and carry information where it may be known whether the second electronic device is carried by the user) from the second electronic device 401 and determining that the second activity state indicating the user activity state of the second electronic device 401 is the walking state based on the information for determining the walking state, the first electronic device 301 may update the user activity state of the first electronic device 301 to the walking state.

For example, upon receiving the information for determining the biking state (e.g., information indicating that the second electronic device is located outdoors and moving speed information about the second electronic device) from the second electronic device 401 and determining that the second activity state indicating the user activity state of the second electronic device is the biking state based on the information for determining the biking state, the first electronic device 301 may update the user activity state of the first electronic device 301 to the biking state.

For example, upon receiving the information for determining the vehicle boarding state (e.g., at least one of vibration pattern information about the vehicle, magnetic pattern information corresponding to the traveling state of the vehicle, or engine output sound information about the vehicle and moving speed information about the second electronic device) from the second electronic device 401 and determining that the second activity state indicating the user activity state of the second electronic device is the vehicle boarding state based on the information for determining the vehicle boarding state, the first electronic device 301 may update the user activity state of the first electronic device 301 to the vehicle boarding state.

According to an embodiment, as the first electronic device 301 and the second electronic device 401 are synchronized with the same activity state of the user, information collected based on the sensor data obtained through the sensor module included in each electronic device (e.g., the first electronic device 301 and the second electronic device 401) may be shared during the synchronized activity state of the user.

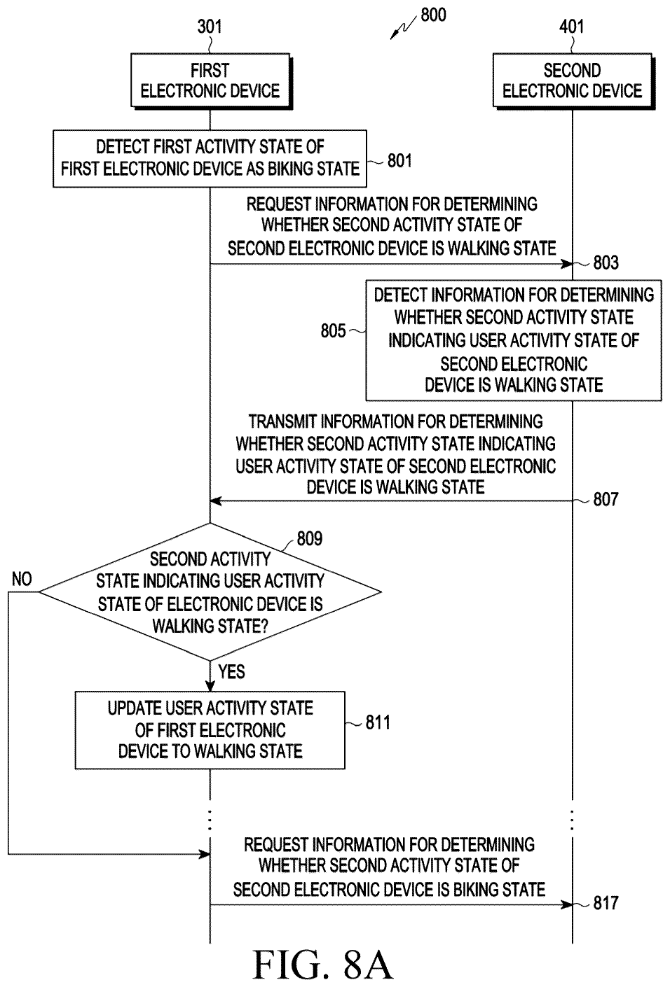

FIG. 7 is a flowchart 700 illustrating an operation of detecting a user activity state in a second electronic device according to an embodiment of the disclosure. The operations of determining the user activity state may include operations 701 to 715. In some embodiments, each operation may be performed sequentially, but is not necessarily required to be performed sequentially. For example, the order of the operations may be changed, and at least two operations may be performed in parallel or other operations may be added.

According to an embodiment, operations 701 to 715 may be understood as performed by the processor (e.g., the second processor 420 of FIG. 4) of the second electronic device 401 (e.g., the second electronic device 401 of FIG. 2 and/or the second electronic device 401 of FIG. 4).

In operation 701, the second electronic device (e.g., the second electronic device 401 of FIG. 2 and/or the second electronic device 401 of FIG. 4) may receive a request for identifying the first activity state of the first electronic device or a request for the user's activity state detected by the second electronic device from the first electronic device 301 (e.g., the first electronic device 301 of FIG. 2 and/or the first electronic device 301 of FIG. 3).

According to an embodiment, the second electronic device may receive the request for identifying the first activity state indicating the user activity state of the first electronic device or the request for the user's activity state detected by the second electronic device from the first electronic device connected through the communication module (e.g., the second communication module 490 of FIG. 4) of the second electronic device.

According to an embodiment, additionally, the second electronic device may receive the request for the information for determining the second activity state indicating the user activity state of the second electronic device from the first electronic device connected through the communication module (e.g., the second communication module 490 of FIG. 4) of the second electronic device.

In operation 703, the second electronic device (e.g., the second electronic device 401 of FIG. 2 and/or the second electronic device 401 of FIG. 4) may identify whether a walking step occurs.

According to an embodiment, the second electronic device may detect a single vector magnitude (SVM) signal from sensor data obtained from the acceleration sensor of the sensor module (e.g., the second sensor module 476 of FIG. 4) of the second electronic device and identify whether a designated walking pattern occurs from the detected signal, determining whether a walking step occurs.

In operation 703, if the occurrence of a walking step is not identified, in operation 705, the second electronic device may identify whether at least one of vibration pattern information about the vehicle, magnetic pattern information corresponding to the traveling state of the vehicle, or engine output sound information about the vehicle is detected, and the moving speed of the second electronic device exceeds a second threshold speed (e.g., about 25 km/h).

According to an embodiment, the second electronic device may detect the vibration pattern information about the vehicle using the acceleration sensor included in the sensor module (e.g., the second sensor module 476 of FIG. 4) of the second electronic device.

According to an embodiment, the second electronic device may detect the magnetic pattern information indicating the traveling state of the vehicle using the geomagnetic sensor included in the sensor module (e.g., the second sensor module 476 of FIG. 4) of the second electronic device.

According to an embodiment, the second electronic device may detect the engine output sound information about the vehicle or a low-frequency signal through the sound sensor (e.g., a microphone) included in the sensor module (e.g., the second sensor module 476 of FIG. 4) of the second electronic device.

According to an embodiment, the second electronic device may identify that the moving speed of the second electronic device exceeds the second threshold speed (e.g., about 25 km/h) using the GPS communication module included in the communication module (e.g. the second communication module 490 of FIG. 4) of the second electronic device.

In operation 705, the second electronic device may detect the at least one of the vibration pattern information about the vehicle, magnetic pattern information corresponding to the traveling state of the vehicle, or engine output sound information about the vehicle using at least one of the sensor module (e.g., the second sensor module 476 of FIG. 4) or the communication module (e.g., the second communication module 490 of FIG. 4) and, upon identifying that the moving speed of the second electronic device exceeds the second threshold speed (e.g., about 25 km/h), identify that the second activity state indicating the user activity state of the second electronic device is the vehicle boarding state in operation 707.

According to an embodiment, the second electronic device may transmit, to the first electronic device, the vehicle boarding state as the second activity state indicating the user activity state of the second electronic device according to the request for identifying the first activity state of the first electronic device received from the first electronic device or the request for the user's activity state detected by the second electronic device.

According to an embodiment, the second electronic device may transmit, to the first electronic device, the vehicle boarding state as the second activity state indicating the user activity state of the second electronic device according to the request for the information for determining the second activity state indicating the user activity state of the second electronic device received from the first electronic device.

In operation 705, if at least one of the vibration pattern information about the vehicle, magnetic pattern information corresponding to the traveling state of the vehicle, or engine output sound information about the vehicle is not detected using at least one of the sensor module (e.g., the second sensor module 476 of FIG. 4) or the communication module (e.g., the second communication module 490 of FIG. 4) or the moving speed of the second electronic device is not identified to exceed the second threshold speed, the second electronic device 401 may identify whether the second electronic device is located outdoors, the moving speed of the second electronic device meets the range of the first threshold speed (e.g., about 10 km/h) or more and the second threshold speed (e.g., about 25 km/h) or less which is the threshold speed range in operation 709.

According to an embodiment, the second electronic device may determine that the second electronic device is located outdoors if the accuracy (e.g., a GPS accuracy value indicating an error range) of location information measured through the GPS communication module included in the communication module (e.g., the second communication module 490 of FIG. 4) of the second electronic device is a predetermined threshold or less (e.g., about 10 m or less or about 20 m or less), the number of satellites caught by the GPS communication module is a predetermined number or more (e.g., three or more), or the RSSI signal of the Wi-Fi communication module included in the communication module (e.g., the second communication module 490 of FIG. 4) of the second electronic device is a predetermined threshold or less (e.g., about −70 dBm or less).

According to an embodiment, the second electronic device may identify the moving speed of the second electronic device through the GPS communication module included in the communication module (e.g., the second communication module 490 of FIG. 4) of the second electronic device, identifying whether it is included in the threshold speed range (e.g., the bikeable speed).

In operation 709, upon identifying that the second electronic device is located outdoors, and the moving speed of the second electronic device meets the range of the first threshold speed (e.g., about 10 km/h) or more and the second threshold speed (e.g., about 25 km/h) or less which is the threshold speed range, the second electronic device may identify the second activity state indicating the user activity state of the second electronic device as the biking state in operation 711.

According to an embodiment, the second electronic device may transmit, to the first electronic device, the biking state as the second activity state indicating the user activity state of the second electronic device according to the request for identifying the first activity state of the first electronic device received from the first electronic device or the request for the user's activity state detected by the second electronic device.

According to an embodiment, the second electronic device may transmit, to the first electronic device, the biking state as the second activity state indicating the user activity state of the second electronic device according to the request for the information for determining the second activity state indicating the user activity state of the second electronic device received from the first electronic device.

In operation 709, upon identifying that the second electronic device is not located outdoors, and the moving speed of the second electronic device is less than the first threshold speed range (e.g., about 10 km/h), the second electronic device may identify the second activity state indicating the user activity state of the second electronic device as the walking state in operation 715.

In operation 703, upon identifying the occurrence of a walking step, the second electronic device may identify that the second electronic device is carried by the user in operation 713. According to an embodiment, upon obtaining the user's biological information (e.g., an electrical signal) using the sensor module of the second electronic device, the second electronic device 401 may identify that the second electronic device is carried in the user's hand.

According to an embodiment, the second electronic device 401 may identify that the second electronic device is carried in the user's hand using a grip sensor among the sensor modules of the second electronic device.

For example, the second electronic device 401 may identify that the second electronic device is carried in a garment (e.g., a pocket of an outer garment) and/or an accessory (e.g., a bag) worn by the user using an inertia sensor among sensor modules of the second electronic device.

In operation 713, upon identifying that the second electronic device is carried by the user, the second electronic device may identify the second activity state indicating the user activity state of the second electronic device as the walking state in operation 715. According to an embodiment, the second electronic device may transmit, to the first electronic device, the walking state as the second activity state indicating the user activity state of the second electronic device according to the request for identifying the first activity state of the first electronic device received from the first electronic device or the request for the user's activity state detected by the second electronic device.

According to an embodiment, the second electronic device may transmit, to the first electronic device, the walking state as the second activity state indicating the user activity state of the second electronic device according to the request for the information for determining the second activity state indicating the user activity state of the second electronic device received from the first electronic device.

In operation 713, upon failing to identify that the second electronic device is carried by the user, the second electronic device may perform operation 709.

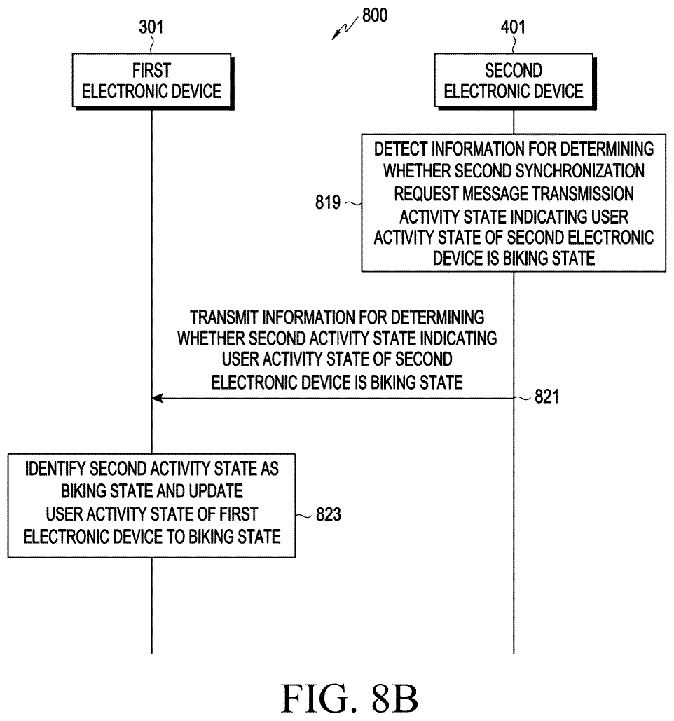

FIGS. 8A and 8B are flowcharts 800 illustrating an operation of determining a user activity state using a plurality of electronic devices according to an embodiment of the disclosure. The operations of determining the user activity state may include operations 801 to 823. In some embodiments, each operation may be performed sequentially, but is not necessarily required to be performed sequentially. For example, the order of the operations may be changed, and at least two operations may be performed in parallel or other operations may be added.

According to an embodiment, operations 801 to 823 may be understood as performed by the processor (e.g., the first processor 320 of FIG. 3 or the second processor 420 of FIG. 4) of each electronic device (e.g., the first electronic device 301 or the second electronic device 401).

In operation 801, the first electronic device 301 (e.g., the first electronic device 301 of FIG. 2 and/or the first electronic device 301 of FIG. 3) may detect the first activity state indicating the user activity state of the first electronic device as the biking state.

For example, the first electronic device may detect the first activity state indicating the user activity state of the first electronic device as the biking state using the sensor module (e.g., the first sensor module 376 of FIG. 3) of the first electronic device or the communication module (e.g., the first communication module 390 of FIG. 3) of the first electronic device.

In operation 803, the first electronic device 301 (e.g., the first electronic device 301 of FIG. 2 and/or the first electronic device 301 of FIG. 3) may request information for determining whether the second activity state of the second electronic device is the walking state from the second electronic device 401.

In operation 805, the second electronic device 401 (e.g., the second electronic device 401 of FIG. 2 and/or the second electronic device 401 of FIG. 4) may detect the information for determining whether the second activity state indicating the user activity state of the second electronic device is the walking state.

According to an embodiment, the second electronic device 401 may detect the information for determining whether the second activity state indicating the user activity state of the second electronic device is the walking state (e.g., walking step occurrence information and carry information where it may be known whether the second electronic device is carried by the user) using the sensor module (e.g., the second sensor module 476 of FIG. 4) of the second electronic device.

In operation 807, the second electronic device 401 (e.g., the second electronic device 401 of FIG. 2 and/or the second electronic device 401 of FIG. 4) may transmit, to the first electronic device, the information for determining whether the second activity state indicating the user activity state of the second electronic device is the walking state.

In operation 809, the first electronic device 301 (e.g., the first electronic device 301 of FIG. 2 and/or the first electronic device 301 of FIG. 3) may determine whether the second activity state indicating the user activity state of the second electronic device is the walking state.

According to an embodiment, the first electronic device 301 may determine whether the second activity state indicating the user activity state of the second electronic device is the walking state based on the information for determining whether it is the walking state received from the second electronic device.

In operation 809, upon determining that the second activity state indicating the user activity state of the second electronic device is the walking state, the first electronic device may update the user activity state of the first electronic device to the walking state in operation 811.

In operation 809, upon failing to determine that the second activity state indicating the user activity state of the second electronic device is the walking state, the first electronic device 301 may request information for determining whether the second activity state of the second electronic device is the biking state from the second electronic device 401 in operation 817.

In operation 819, the second electronic device 401 (e.g., the second electronic device 401 of FIG. 2 and/or the second electronic device 401 of FIG. 4) may detect the information for determining whether the second activity state indicating the user activity state of the second electronic device is the biking state.

According to an embodiment, the second electronic device 401 may detect the information for determining whether the second activity state indicating the user activity state of the second electronic device is the biking state (e.g., information indicating that the second electronic device is located outdoors and moving speed information about the second electronic device) using at least one of the sensor module (e.g., the second sensor module 476 of FIG. 4) of the second electronic device or the communication module (e.g., the second communication module 490 of FIG. 4) of the second electronic device.

In operation 821, the second electronic device 401 (e.g., the second electronic device 401 of FIG. 2 and/or the second electronic device 401 of FIG. 4) may transmit, to the first electronic device 301, the information for determining whether the second activity state indicating the user activity state of the second electronic device is the biking state.

In operation 823, upon identifying that the second activity state indicating the user activity state of the second electronic device is the biking state, the first electronic device 301 (e.g., the first electronic device 301 of FIG. 2 and/or the first electronic device 301 of FIG. 3) may update the user activity state of the first electronic device to the biking state.

According to an embodiment, the first electronic device 301 may determine that the second activity state indicating the user activity state of the second electronic device is the biking state based on the information for determining whether it is the biking state received from the second electronic device.

In FIGS. 8A and 8B, an example is described in which the first activity state of the first electronic device is detected as the biking state, but the first activity state of the first electronic device may be detected as the walking state or the vehicle boarding state. In FIGS. 8A and 8B, an example is described in which the second activity state of the second electronic device is detected as the walking state, but the second activity state of the second electronic device may be detected as the biking state or the vehicle boarding state.

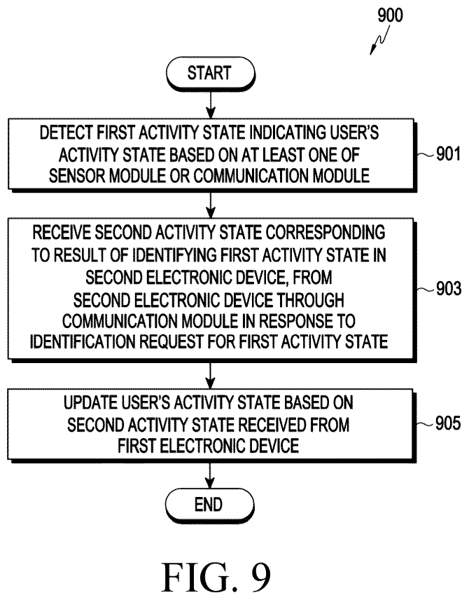

FIG. 9 is a flowchart 900 illustrating an operation of determining a user activity state in a first electronic device according to an embodiment of the disclosure. The operations of determining the user activity state may include operations 901 to 905. In some embodiments, each operation may be performed sequentially, but is not necessarily required to be performed sequentially. For example, the order of the operations may be changed, and at least two operations may be performed in parallel or other operations may be added.

According to an embodiment, operations 901 to 905 may be understood as performed by the processor (e.g., the first processor 320 of FIG. 3) of the first electronic device (e.g., the first electronic device 301 of FIG. 2 and/or the first electronic device 301 of FIG. 3). In operation 901, the first electronic device 301 (e.g., the first electronic device 301 of FIG. 2 and/or the first electronic device 301 of FIG. 3) may detect the first activity state indicating the user's activity state based on at least one of the sensor module or the communication module.

According to an embodiment, the first electronic device may detect the first activity state indicating the activity state of the user of the first electronic device based on at least one sensor (e.g., an acceleration sensor and/or a gyro sensor) among the plurality of sensors included in the sensor module (e.g., the first sensor module 376 of FIG. 3) of the first electronic device or at least one communication module (e.g., a GPS communication module) among the plurality of communication modules included in the communication module (e.g., the first communication module 390 of FIG. 3) of the first electronic device.

According to an embodiment, the first activity state may include a walking state, a biking state, or a vehicle boarding state.

In operation 903, the first electronic device 301 (e.g., the first electronic device 301 of FIG. 2 and/or the first electronic device 301 of FIG. 3) may receive, from the second electronic device (e.g., the second electronic device 401 of FIG. 2 and/or the second electronic device 401 of FIG. 4) through the communication module, the second activity state corresponding to the result of identifying the first activity state in the second electronic device in response to the identification request for the first activity state.

According to an embodiment, they may be connected through short-range communication using the communication module (e.g., the first communication module 390 of FIG. 3) of the first electronic device and the communication module (e.g., the second communication module 490 of FIG. 4) of the second electronic device.

According to an embodiment, the first electronic device may request the second electronic device to identify the first activity state through the communication module (e.g., the first communication module 390 of FIG. 3) of the first electronic device.

According to an embodiment, the first electronic device may receive the second activity state corresponding to the result of identifying the first activity state of the first electronic device from the second electronic device through the communication module (e.g., the first communication module 390 of FIG. 3) of the first electronic device.

According to an embodiment, the second activity state may include a walking state, a biking state, or a vehicle boarding state.

In operation 905, the first electronic device 301 (e.g., the first electronic device 301 of FIG. 2 and/or the first electronic device 301 of FIG. 3) may update the user's activity state based on the second activity state received from the first electronic device.

According to an embodiment, when the second activity state (e.g., the biking state) received from the second electronic device is identical to the first activity state (e.g., the biking state) detected by the first electronic device 301, the first electronic device may update the first activity state (e.g., the biking state) to the user's activity state. For example, upon updating the user activity state of the first electronic device to the biking state, the first electronic device may operate a fall detection algorithm used in the biking state.

According to an embodiment, when the second activity state (e.g., the vehicle boarding state) received from the second electronic device is different from the first activity state (e.g., the bicycle boarding state) detected by the first electronic device 301, the first electronic device may update the second activity state (e.g., the vehicle boarding state) to the user's activity state. For example, upon updating the user activity state of the first electronic device to the vehicle boarding state, the first electronic device may operate a vehicle collision accident detection algorithm.

According to an embodiment, a method for operating an electronic device may include requesting, by a first electronic device, a second electronic device to identify a first activity state detected as a user's activity state, identifying, by the second electronic device, whether the user's activity state is the first activity state upon receiving the request for identifying the first activity state from the first electronic device, transmitting, by the second electronic device, the second activity state corresponding to the identification result to the first electronic device, and updating, by the first electronic device, the activity state of the user based on a second activity state corresponding to an identification result of identifying the first activity state in the second electronic device received from the second electronic device in response to the request.

According to an embodiment, the first electronic device may include a wearable electronic device.

According to an embodiment, the method may further include detecting, by the first electronic device, the first activity state based on at least one of a sensor module or a communication module included in the first electronic device.

According to an embodiment, the method may further include updating, by the first electronic device, the first activity state to the user's activity state if the second activity state received from the first electronic device is identical to the first activity state.

According to an embodiment, the method may further include updating, by the first electronic device, the second activity state to the user's activity state if the second activity state received from the first electronic device is different from the first activity state.

According to an embodiment, the method may further include, upon identifying that a walking step occurs, and the second electronic device is carried by the user using a sensor module of the second electronic device, detecting, by the second electronic device, the user's walking state as the second activity state, upon identifying that the second electronic device is located indoor, and a moving speed of the second electronic device meets a threshold speed range using at least one of the sensor module or communication module of the second electronic device, detecting, by the second electronic device, a biking state as the second activity state, and detecting, by the second electronic device, at least one of vibration pattern information about the vehicle, magnetic pattern information corresponding to a traveling state of the vehicle, or vehicle engine output sound information using at least one of the sensor module or the communication module of the second electronic device and, upon identifying that the moving speed of the second electronic device exceeds a second threshold speed, detect a vehicle boarding state as the second activity state.

According to an embodiment, a method for determining a user's activity state in an electronic device may include detecting a first activity state indicating the user's activity state based on at least one of a sensor module of the electronic device or a communication module of the electronic device, receiving a second activity state corresponding to a result of identifying the first activity state in a first electronic device from the first electronic device through the communication module in response to a request for identifying the first activity state, and updating the user's activity state based on a second activity state received from the first electronic device.

According to an embodiment, the method may further include updating the first activity state to the user's activity state if the second activity state received from the first electronic device is identical to the first activity state and updating the second activity state to the user's activity state if the second activity state received from the first electronic device is different from the first activity state.

According to an embodiment, a method for operating an electronic device may include, upon receiving a request for identifying a first activity state from a first electronic device, detecting a second activity state corresponding to a result of identifying whether a user's activity state is the first activity state based on at least one of the sensor module or the communication module, and transmitting the second activity state to the first electronic device through the communication module of the electronic device.

According to an embodiment, the method may further include, upon identifying that a walking step occurs, and the second electronic device is carried by the user using a sensor module, detecting the user's walking state as the second activity state, upon identifying that the second electronic device is located indoor, and a moving speed of the second electronic device meets a threshold speed range using at least one of the sensor module or communication module, detecting a biking state as the second activity state, and detecting at least one of magnetic pattern information or vehicle engine output sound information corresponding to a traveling state of the vehicle and vibration pattern information about the vehicle using at least one of the sensor module or communication module and, upon identifying that the moving speed of the second electronic device exceeds a second threshold speed, detect a vehicle boarding state as the second activity state.

According to embodiments of the disclosure, as more accurate user activity state is recognized using a plurality of electronic devices, it is possible to increase the performance of the algorithm capable of recognizing the user's risk.

The electronic device according to an embodiment of the disclosure may be one of various types of electronic devices. The electronic devices may include, for example, a portable communication device (e.g., a smartphone), a computer device, a portable multimedia device, a portable medical device, a camera, a wearable device, or a home appliance. According to an embodiment of the disclosure, the electronic devices are not limited to those described above.

It should be appreciated that various embodiments of the present disclosure and the terms used therein are not intended to limit the technological features set forth herein to particular embodiments and include various changes, equivalents, or replacements for a corresponding embodiment. With regard to the description of the drawings, similar reference numerals may be used to refer to similar or related elements. It is to be understood that a singular form of a noun corresponding to an item may include one or more of the things, unless the relevant context clearly indicates otherwise. As used herein, each of such phrases as “A or B,” “at least one of A and B,” “at least one of A or B,” “A, B, or C,” “at least one of A, B, and C,” and “at least one of A, B, or C,” may include all possible combinations of the items enumerated together in a corresponding one of the phrases. As used herein, such terms as “1st” and “2nd,” or “first” and “second” may be used to simply distinguish a corresponding component from another, and does not limit the components in other aspect (e.g., importance or order). It is to be understood that if an element (e.g., a first element) is referred to, with or without the term “operatively” or “communicatively”, as “coupled with,” “coupled to,” “connected with,” or “connected to” another element (e.g., a second element), it means that the element may be coupled with the other element directly (e.g., wiredly), wirelessly, or via a third element.

As used herein, the term “module” may include a unit implemented in hardware, software, or firmware, and may interchangeably be used with other terms, for example, “logic,” “logic block,” “part,” or “circuitry”. A module may be a single integral component, or a minimum unit or part thereof, adapted to perform one or more functions. For example, according to an embodiment, the module may be implemented in a form of an application-specific integrated circuit (ASIC).

An embodiment of the disclosure may be implemented as software (e.g., the program 140) including one or more instructions that are stored in a storage medium (e.g., internal memory 136 or external memory 138) that is readable by a machine (e.g., the electronic device 101 or the electronic device 301). For example, a processor (e.g., the processor 520) of the machine (e.g., the electronic device 301) may invoke at least one of the one or more instructions stored in the storage medium, and execute it, with or without using one or more other components under the control of the processor. This allows the machine to be operated to perform at least one function according to the at least one instruction invoked. The one or more instructions may include a code generated by a complier or a code executable by an interpreter. The storage medium readable by the machine may be provided in the form of a non-transitory storage medium. Wherein, the term “non-transitory” simply means that the storage medium is a tangible device, and does not include a signal (e.g., an electromagnetic wave), but this term does not differentiate between where data is semi-permanently stored in the storage medium and where the data is temporarily stored in the storage medium.

According to an embodiment, a method according to various embodiments of the disclosure may be included and provided in a computer program product. The computer program products may be traded as commodities between sellers and buyers. The computer program product may be distributed in the form of a machine-readable storage medium (e.g., compact disc read only memory (CD-ROM)), or be distributed (e.g., downloaded or uploaded) online via an application store (e.g., Play Store™), or between two user devices (e.g., smartphones) directly. If distributed online, at least part of the computer program product may be temporarily generated or at least temporarily stored in the machine-readable storage medium, such as memory of the manufacturer's server, a server of the application store, or a relay server.

According to an embodiment, each component (e.g., a module or a program) of the above-described components may include a single entity or multiple entities. Some of the plurality of entities may be separately disposed in different components. According to an embodiment, one or more of the above-described components may be omitted, or one or more other components may be added. Alternatively or additionally, a plurality of components (e.g., modules or programs) may be integrated into a single component. In such a case, according to various embodiments, the integrated component may still perform one or more functions of each of the plurality of components in the same or similar manner as they are performed by a corresponding one of the plurality of components before the integration. According to various embodiments, operations performed by the module, the program, or another component may be carried out sequentially, in parallel, repeatedly, or heuristically, or one or more of the operations may be executed in a different order or omitted, or one or more other operations may be added.

According to an embodiment of the disclosure, in a non-volatile storage medium storing instructions, the instructions are configured to, when executed by an electronic device, enable the electronic device to perform at least one operation, and the at least one operation may include requesting, by a first electronic device, a second electronic device to identify a first activity state detected as a user's activity state, updating, by the first electronic device, the user's activity state based on a second activity state corresponding to a result of identifying the first activity state in a second electronic device received from the second electronic device in response to the request, identifying, by the second electronic device, whether the user's activity state is the first activity state upon receiving the request for identifying the first activity state from the first electronic device, and transmitting, by the second electronic device, the second activity state corresponding to the identification result to the first electronic device.

According to an embodiment of the disclosure, in a non-volatile storage medium storing instructions, the instructions are configured to, when executed by an electronic device, enable the electronic device to perform at least one operation, and the at least one operation may include detecting a first activity state indicating the user's activity state based on at least one of a sensor module of the electronic device or a communication module of the electronic device, receiving a second activity state corresponding to a result of identifying the first activity state in a first electronic device from the first electronic device through the communication module in response to a request for identifying the first activity state, and updating the user's activity state based on a second activity state received from the first electronic device.

According to an embodiment of the disclosure, in a non-volatile storage medium storing instructions, the instructions are configured to, when executed by an electronic device, enable the electronic device to perform at least one operation, and the at least one operation may include, upon receiving a request for identifying a first activity state from a first electronic device, detecting a second activity state corresponding to a result of identifying whether a user's activity state is the first activity state based on at least one of the sensor module or the communication module, and transmitting the second activity state to the first electronic device through the communication module of the electronic device.

The above-described embodiments are merely specific examples to describe technical content according to the embodiments of the disclosure and help the understanding of the embodiments of the disclosure, not intended to limit the scope of the embodiments of the disclosure. Accordingly, the scope of various embodiments of the disclosure should be interpreted as encompassing all modifications or variations derived based on the technical spirit of various embodiments of the disclosure in addition to the embodiments disclosed herein.

What is claimed is:

1. A system for determining an activity state of a user, the system comprising:
a first electronic device; and
a second electronic device,
wherein the first electronic device is configured to:
request the second electronic device to identify a first activity state detected as an activity state of a user in the first electronic device,
receive a second activity state corresponding to an identification result of identifying the first activity state in the second electronic device received from the second electronic device in response to the request, and
update the activity state of the user based on the second activity state, and
wherein the second electronic device is configured to:
based on receiving the request for identifying the first activity state from the first electronic device, identify whether the activity state of the user is the first activity state and transmit the second activity state corresponding to the identification result to the first electronic device.

2. The system of claim 1, wherein the first electronic device comprises a wearable electronic device.

3. The system of claim 1, wherein the first electronic device is further configured to detect the first activity state based on at least one of a sensor module or a communication module included in the first electronic device.

4. The system of claim 1, wherein the first electronic device is further configured to update the activity state of the user to the first activity state based on the second activity state received from the second electronic device corresponding to the first activity state.

5. The system of claim 1, wherein the first electronic device is further configured to update the activity state of the user to the second activity state based on the second activity state received from the second electronic device being different from the first activity state.

6. The system of claim 1, wherein the second electronic device is further configured to:
based on identifying that a walking step occurs and the second electronic device is carried by the user using a sensor module of the second electronic device, detect a walking state of the user as the second activity state;
based on identifying that the second electronic device is located indoor and a moving speed of the second electronic device meets a threshold speed range using at least one of the sensor module or communication module of the second electronic device, detect a biking state as the second activity state; and
detect at least one of vibration pattern information about a vehicle, vehicle engine output sound information, or magnetic pattern information corresponding to a traveling state of the vehicle using at least one of the sensor module or the communication module of the second electronic device and, based on identifying that the moving speed of the second electronic device exceeds a second threshold speed, detect a vehicle boarding state as the second activity state.

7. An electronic device comprising:
a sensor module;
a communication module;
at least one processor, and
memory storing instructions that, when executed by the at least one processor individually or collectively, cause the electronic device to:
detect a first activity state indicating an activity state of a user based on at least one of the sensor module or the communication module;
receive a second activity state corresponding to a result of identifying the first activity state in a first electronic device from the first electronic device through the communication module in response to a request for identifying the first activity state; and
update the activity state of the user based on the second activity state received from the first electronic device.

8. The electronic device of claim 7, wherein the instructions, when executed by the at least one processor individually or collectively, cause the wearable electronic device to:
update the activity state of the user to the first activity state based on the second activity state received from the first electronic device corresponding to the first activity state; and
update the activity state of the user to the second activity state based on the second activity state received from the first electronic device being different from the first activity state.

9. An electronic device comprising:
a communication module;
a sensor module;
at least one processor; and
memory storing instructions that, when executed by the at least one processor individually or collectively, cause the electronic device to:
based on receiving a request for identifying a first activity state from a first electronic device, detect a second activity state corresponding to a result of identifying whether an activity state of a user is the first activity state based on at least one of the sensor module or the communication module; and
transmit the second activity state to the first electronic device through the communication module.

10. The electronic device of claim 9, wherein the instructions, when executed by the at least one processor individually or collectively, cause the wearable electronic device to:
based on identifying that a walking step occurs and the electronic device is carried by the user using the sensor module, detect a walking state of the user as the second activity state;
based on identifying that the electronic device is located indoor and a moving speed of the second electronic device meets a threshold speed range using at least one of the sensor module or the communication module, detect a biking state as the second activity state;
detect at least one of vibration pattern information about a vehicle, magnetic pattern information corresponding to a traveling state of the vehicle, or vehicle engine output sound information using at least one of the sensor module or the communication module; and
based on identifying that the moving speed of the electronic device exceeds a second threshold speed, detect a vehicle boarding state as the second activity state.

11. A method for operating an electronic device, the method comprising:
requesting, by a first electronic device, a second electronic device to identify a first activity state detected as an activity state of a user;
identifying, by the second electronic device, whether the activity state of the user is the first activity state based on receiving the request for identifying the first activity state from the first electronic device;
transmitting, by the second electronic device, the second activity state corresponding to the identification result to the first electronic device, and
updating, by the first electronic device, the activity state of the user based on a second activity state corresponding to an identification result of identifying the first activity state in the second electronic device received from the second electronic device in response to the request.

12. The method of claim 11, further comprising detecting, by the first electronic device, the first activity state based on at least one of a sensor module or a communication module included in the first electronic device.

13. The method of claim 11, further comprising updating, by the first electronic device, the activity state of the user to the first activity state based on the second activity state received from the first electronic device corresponding to the first activity state.

14. The method of claim 11, further comprising updating, by the first electronic device, the activity state of the user to the second activity state based on the second activity state received from the first electronic device being different from the first activity state.

15. The method of claim 11, further comprising:
based on identifying that a walking step occurs and the second electronic device is carried by the user using a sensor module of the second electronic device, detecting, by the second electronic device, the a walking state of the user as the second activity state;
based on identifying that the second electronic device is located indoor and a moving speed of the second electronic device meets a threshold speed range using at least one of the sensor module or communication module of the second electronic device, detecting, by the second electronic device, a biking state as the second activity state; and
detecting, by the second electronic device, at least one of vibration pattern information about a vehicle, magnetic pattern information corresponding to a traveling state of the vehicle, or vehicle engine output sound information using at least one of the sensor module or the communication module of the second electronic device and, based on identifying that the moving speed of the second electronic device exceeds a second threshold speed, detect a vehicle boarding state as the second activity state.
